(19) United States
(12) Patent Application Publication
(10) Pub. No.: US 2024/0198066 A1
(43) Pub. Date: Jun. 20, 2024

(54) SYSTEM CONTAINING INGESTIBLE ULTRASOUND DEVICE FOR DELIVERY OF THERAPEUTIC AGENTS

(71) Applicant: Suono Bio, Inc., Foxborough, MA (US)

(72) Inventors: Carl Schoellhammer, Foxborough, MA (US); Scott Kellogg, Mattapoisett, MA (US); Albert Farinha, Barrington, RI (US)

(21) Appl. No.: 18/287,086

(22) PCT Filed: Apr. 15, 2022
(86) PCT No.: PCT/US2022/024987

§ 371 Date: Oct. 16, 2023

Related U.S. Application Data
(60) Provisional application No. 63/176,643, filed on Apr. 19, 2021.

(51) Int. Cl. A61M 31/00; A61M 37/00

(52) U.S. Cl. CPC A61M 31/002; A61M 37/0092; A61M 2205/055; A61M 2205/3523; A61M 2205/502; A61M 2210/1042

(57) ABSTRACT

The invention provides systems and components thereof for ultrasound-mediated drug delivery within the GI tract delivery of therapeutic agents to the gastrointestinal tract of a subject. The systems comprise a transmitter that includes a power source and power emitter for transmitting EM and non-EM energy. The power transmitter comprises one or more capacitive, inductive, magnetic resonance. RF, or ultrasonic energy radiator. The second component is an ingestible capsule that is physically separate from the transmitter. The ingestible capsule may be configured to harvest energy from the transmitter using a variety of components. In various embodiments, the ingestible pill comprises at least one energy harvesting component, an ultrasound transducer, and a reservoir or payload that releasably retains a liquid or powder mixture containing an encapsulated or non-encapsulated therapeutic agent. When in use, the ingestible capsule enters the GI tract of the subject, while the transmitter remains external to the subject's body.

### CROSS-REFERENCE TO RELATED APPLICATIONS

The present application claims priority to and the benefit of U.S. Provisional Application Ser. No. 63/176,643, filed on Apr. 19, 2021, the content of which is incorporated herein by reference in its entirety.

### FIELD OF THE INVENTION

The invention relates generally to devices and methods for ultrasonic delivery of an agent to an internal tissue.

### BACKGROUND

The most common route of drug delivery is oral administration. Many drugs can be readily absorbed in the gastrointestinal (GI) tract, so oral administration allows them to enter the blood quickly and circulate systemically. In addition, oral administration is convenient and minimally invasive.

Nonetheless, oral administration is not suitable for all drugs. For some drugs, the acidic conditions and harsh digestive enzymes of the GI tract degrade or inactivate the active pharmaceutical ingredient (API) before it can reach its target tissue. Other therapeutic agents, such as biological therapeutics (“biologics”), which generally consist of large macromolecules, are poorly absorbed in the GI tract. Absorption may also be limited if the patient has diarrhea, which minimizes the duration of transit of the drug through the GI tract.

Ingestible ultrasonic drug delivery devices have been developed to overcome the difficulty of delivering certain drugs via the GI tract. Such devices include an ultrasound transducer, a reservoir that stores the drug, and a power source, such as a battery and drive circuitry, that drives the transducer. However, the utility of these fully self-contained devices is limited by a different set of technical obstacles. For example, the device must be small enough that it can be easily swallowed, yet large enough to accommodate the drug, transducer, drive circuitry, and battery. These factors constrain the quantity of drug that can be delivered by all-in-one ingestible ultrasonic drug delivery devices. Another consideration is that the battery can severely damage internal tissue if it makes electrical contact with the tissue. Therefore, the device must contain material to electrically insulate the battery, which further restricts the size and drug-loading capacity of the device. Consequently, these factors largely limit the therapeutic potential of drug delivery via ingestible ultrasonic devices.

### SUMMARY

The invention provides systems that may comprise an ingestible capsule that includes an ultrasound transducer, wireless power/energy, transfer/harvester (WPTH) device, a drug payload or drug reservoir and a separate power/energy transmitter that can control or power the ingestible capsule device remotely. During use, the ingestible capsule is positioned within the subject's gastrointestinal (GI) tract, while the transmitter remains external to the subject's body. The design of a system in which the ultrasound transducer is separate from the transmitter, including the power source, obviates the need to include a battery and drive circuitry in the form-factor that must be swallowed. Consequently, the system allows the ingestible capsule device to be smaller and/or have a greater drug payload than prior self-contained ingestible ultrasound devices. In addition, the two-component systems eliminate the risk associated with passage of a battery-containing device through a person's GI tract.

Certain aspects of the present disclosure may include an ingestible capsule drug delivery system for targeted or localized ultrasound-mediated drug delivery within the GI tract. In various embodiments, the ingestible capsule may comprise one or more wireless energy harvesters, antennae, impedance matching networks, rectifiers, voltage multipliers, charge controllers, energy storage devices, ultrasound transducer drivers, ultrasound transducers, drug carrier/reservoirs, and at least one drug payload containing at least one therapeutic agent. In various embodiments, the wireless energy harvester can comprise one or more components for wireless power transfer (WPT) or energy harvesting. In various embodiments, the WPT may comprise electromagnetic (EM) methods, including but not limited to, capacitive coupling, magnetic resonance, inductive coupling, inductive energy transfer, mid-field radiative/non-radiative, and radiative far-field. In various embodiments, the WPT can comprise non-EM methods, preferably acoustic or ultrasound (US) using piezoelectric structures to convert US vibration into electric energy or power. In various embodiments, one or more ingestible capsule functions may be controlled by an external power/energy transmitter, including but not limited to, energy transfer to at least one of the capsule's energy harvester, data transmission, activation of the drug carrier or reservoir, activation of the drug payload, activation of a therapeutic agent, or modification of the local external environment of the capsule within the GI tract. Activation may be in the form of capacitive, inductive coupling, inductive transfer, magnetic resonance, EM radiation, or ultrasonic energy.

Aspects of the present disclosure may include an ingestible capsule that comprises at least one inductive receiver coil configured to receive an EM wave, energy, or signal from a transmitter external to the capsule, an ultrasound transducer electrically coupled to the inductive receiver, and a reservoir configured to releasably retain a liquid comprising a therapeutic agent. The ingestible capsule does not include a power source. In certain aspects, a reservoir may be configured to releasably retain at least one non-encapsulated or encapsulated therapeutic agent.

Aspects of the present disclosure may comprise an ingestible capsule that includes one or more inductive receiver coils configured to receive an EM wave, energy, or signal from a transmitter external to the capsule; an ultrasound transducer electrically coupled to the inductive receiver; and a reservoir configured to releasably retain a liquid comprising a non-encapsulated or an encapsulated therapeutic agent. In various embodiments, electrical couplings can include one or more, antennae, tuning circuits, AC-DC voltage converters, voltage regulators, or combinations thereof. In various embodiments, said transmitter may comprise one or more antenna, coil, tuning circuit, AC-DC converter, and combinations thereof. In various embodiments, the transmitter may be worn by a person, in the proximity, or placed at a distant location. The ingestible capsule drug delivery system is preferably designed to transfer power with high efficiency and stability.

Aspects of the present invention may include ingestible capsule drug delivery systems comprising a radiofrequency (RF) transmitter containing an antenna and an ingestible capsule containing an RF energy harvester. In various embodiments, the RF energy harvester can comprise an RF receiving antenna, an impedance matching network circuit, RF-DC converter, and an energy storage device. In various embodiments, the energy storage device can provide power for an ultrasound transducer driver or load. In various embodiments, the RF receiving antenna of the capsule may comprise an isotropic or directional antenna. In various embodiments, the impedance matching network may be tuned to maximize power transfer from the receiving antenna to the rectifier circuit. In some embodiments, the RF receiving antenna may be a rectenna (rectifying antenna), rectifying incoming EM waves into DC current.

Certain aspects of the present disclosure may include an ingestible capsule drug delivery system comprising an external transmitter containing a piezoelectric ultrasonic transducer operating in conjunction with an ingestible capsule containing a piezoelectric transducer energy harvester. In various embodiments, the external transmitter can comprise a voltage source, microcontroller, one or more resistors, one or more transistors, an amplifier to drive a piezoelectric transducer or an array of piezoelectric transducers. In various embodiments, the piezoelectric transducer energy harvester can further comprise a power conditioning circuit to convert an AC output voltage into DC voltage to power an ultrasound transducer driver or load within the capsule. In various embodiments, the transmitter may contain one or more piezoelectric transducer configured to operate in one or more mode, including but not limited to, thickness vibration, radial vibration, transverse vibration, flexural, the like, or combination of. In preferred embodiments, the transmitting piezoelectric transducer may be configured to operate in the thickness vibration mode to transfer power with high efficiency and stability. In some embodiments, the external transmitter comprises a piezoelectric transducer to produce non-focused or focused ultrasound at a surface or within the ingestible capsule. In another alternative embodiment, the external transmitter comprises a piezoelectric transducer array made up of n-elements to deliver diffused or focused ultrasound to the ingestible capsule within the GI tract. In various embodiments, the ultrasound transducer may be positioned to transduce ultrasound waves in a particular direction relative to the reservoir of the ingestible capsule. The ultrasound transducer may be positioned to transduce ultrasound waves toward the reservoir. The ultrasound transducer can be positioned to transduce ultrasound waves away from the reservoir. The ultrasound transducer may be positioned to produce omnidirectional ultrasound waves through the reservoir. The reservoir can be configured to releasably retain a liquid comprising a therapeutic agent or an encapsulated therapeutic agent.

Aspects of the invention may include an ingestible capsule containing a modulator that modulates a frequency of an electromagnetic signal received by the said inductive receiver coil. The modulator may be electrically coupled to the inductive receiver coil and the transducer. The modulator may be a multiplier that increases the frequency of the electromagnetic signal received by the inductive receiver coil. The modulator may be an attenuator that decreases the frequency of the electromagnetic signal received by the inductive receiver coil. The ingestible capsule may contain a component that alters a frequency of an electromagnetic signal received by the inductive receiver coil.

The ultrasound transducer of the ingestible capsule may produce an ultrasound signal with a defined frequency or within a defined frequency range. The ultrasound transducer may produce an ultrasound signal of from about 10 kHz to about 10 MHz, from about 10 kHz to about 1 MHz, from about 10 kHz to about 100 kHz, from about 20 kHz to about 80 kHz, from about 20 kHz to about 60 kHz, or from about 30 kHz to about 50 kHz. The ultrasound transducer may produce an ultrasound signal of less than 100 kHz, less than 80 kHz, less than 60 kHz, or less than 50 kHz. The ultrasound transducer may produce an ultrasound signal of about 20 kHz, about 25 kHz, about 30 kHz, about 35 kHz, about 40 kHz, about 45 kHz, about 50 kHz, about 55 kHz, or about 60 kHz.

The ingestible capsule may have a defined size, length, or volume. The ingestible capsule may have the longest dimension of less than about 3.0 cm, about 2.75 cm, about 2.5 cm, about 2.25 cm, about 2.0 cm, about 1.75 cm, or about 1.5 cm. The ingestible capsule may have a transverse dimension of less than about 1.2 cm, about 1.1 cm, about 1.0 cm, about 0.9 cm, or about 0.8 cm.

The ingestible capsule may include additional components. The ingestible capsule may include a rectifier that is electrically coupled to the inductive receiver. The ingestible capsule may include an electrode electrically coupled to the rectifier and in contact with the drug payload or reservoir.

In certain aspects the invention may provide a system that includes an inductive transmitter and an ingestible capsule that is physically separate from the transmitter. The transmitter can include a power source and a transmitter coil electrically coupled to the power source. In certain embodiments, the transmitter coil can comprise a Helmholtz coil. In some embodiments, the transmitter coil may comprise a solenoid coil. The ingestible capsule can include at least one inductive receiver coil configured to receive an electromagnetic signal or energy from the transmitter when the transmitter is not in contact with the ingestible capsule, an ultrasound transducer electrically coupled to the inductive receiver, and a reservoir configured to releasably retain a liquid comprising a therapeutic agent or an encapsulated therapeutic agent. In various embodiments, the power source may be a battery. The power source may produce a DC voltage within a defined range. The power source may produce a DC voltage of from about 1.6 VDC to about 64 VDC, from about 3.2 VDC to about 64 VDC, from about 6.4 VDC to about 32 VDC, from about 1.6 VDC to about 32 VDC, from about 3.2 VDC to about 32 VDC, from about 6.4 VDC to about 32 VDC, from about 1.6 VDC to about 16 VDC, from about 3.2 VDC to about 16 VDC, or from about 6.4 VDC to about 16 VDC.

In various aspects, the inductive transmitter may include a DC-DC converter downstream of the power source and upstream of the transmitter coil. The DC-DC converter may increase the voltage produced by the power source to a voltage within a defined range. The DC-DC converter may increase the voltage produced by the power source to from about 8 VDC to about 80 VDC, from about 16 VDC to about 160 VDC, from about 32 VDC to about 320 VDC, from about 8 VDC to about 80 VDC, from about 16 VDC to about 160 VDC, from about 32 VDC to about 320 VDC, from about 8 VDC to about 80 VDC, from about 16 VDC to about 160 VDC, or from about 32 VDC to about 320 VDC. The DC-DC converter may be connected directly to the transmitter coil without intervening components. The transmitter may include one or more additional components. The transducer may include one or more voltage-controlled oscillator, a FET driver, a FET transistor, a capacitor, inductor, resistor, a user interface configured to receive input from a user, a display, and a microprocessor. The transmitter may be configured to be held in the hand of a person. The transmitter may include, or be a part of, a wearable garment. The transmitter may include, or be a part of, a square, circular, cylindrical framed structure configured to surround or expose energy to a person. The transmitter may include, or be a part of, a square, circular, cylindrical framed structure configured to enable a person to enter partially or whole within its volume. The power source may be rechargeable.

Aspects of the invention may include an ingestible capsule that may contain a modulator that modulates a frequency of an electromagnetic signal received by the inductive receiver coil, as described above or it may lack a component that alters a frequency of an electromagnetic signal received by the inductive receiver coil. In the ingestible capsule, the ultrasound transducer may produce an ultrasound signal within a defined frequency range, as described above. The ingestible capsule may have a defined size or length, as described above. The ingestible capsule may include additional components, such as any of those described above.

In some aspects, methods of the invention may include administering a therapeutic agent to a gastrointestinal tissue of a subject by orally administering to a subject an ingestible capsule that does not include a power source but does include at least one inductive receiver coil, an ultrasound transducer electrically coupled to the inductive receiver, and a reservoir comprising a liquid or a powder mixture comprising a therapeutic agent; and transmitting via a transmitter external to the subject an electromagnetic signal to the ingestible capsule to allow the ultrasound transducer to generate an ultrasound signal or ultrasound energy and thereby deliver the therapeutic agent from the reservoir into gastrointestinal tissue of the subject. The transmitter may include any of the components described above, such as a power source and a transmitter coil electrically coupled to the power source. The electromagnetic signal may be transmitted from the transmitter coil to the inductive receiver coil. The ingestible capsule may include any of the components described above, such as a rectifier electrically coupled to the inductive receiver and an electrode electrically coupled to the rectifier and in contact with the reservoir. Transmission of the electromagnetic signal may generate an electrical signal in the liquid that promotes movement of the therapeutic agent from the reservoir and into the gastrointestinal tissue. The electrical signal may be a DC signal or a DC pulse train. The electrical signal may promote movement of the therapeutic agent by iontophoresis, electrophoresis, electroporation, sonoporation, magnetosonoporation, or ultrasonic cavitation.

In certain embodiments, the external transmitter may generate a magnetic field, a magnetic flux, magnetic field gradient, or magnetic force that positions the ingestible capsule adjacent to the gastrointestinal tissue of the subject. In various embodiments, the transmitter may generate an alternating (AC) magnetic field that activate the release of the therapeutic agent from the reservoir into GI tissue. The frequency of the electromagnetic signal may be about equal to a frequency of the ultrasound signal. The frequency of the electromagnetic signal may not be equal to a frequency of the ultrasound signal. The ultrasound signal may have a defined frequency or a defined frequency range, such as any of those described above. In an alternative embodiment, the ingestible capsule comprises a magnetic component. The magnetic component may comprise a diamagnetic, paramagnetic, superparamagnetic, magnetic, or ferromagnet microparticles or nanoparticles. In various embodiments, one or more permanent magnet may be positioned about a subject to attract the ingestible capsule to a specific location of the GI tract.

In various aspects, methods of the invention can include administering a therapeutic agent to a gastrointestinal tissue of a subject by orally administering to a subject an ingestible capsule that does not include a power source but does include at least one piezoelectric transducer for harvesting ultrasonic energy, an ultrasound transducer electrically coupled to said ultrasound energy harvester, and a reservoir comprising a liquid comprising a therapeutic agent; and transmitting via a transmitter external to the subject an ultrasonic wave to the ingestible capsule to provide power for driving the ultrasound transducer to generate an ultrasound signal and thereby deliver the therapeutic agent from the reservoir into gastrointestinal tissue of the subject.

Aspects of the invention may include methods of administering a therapeutic agent to a gastrointestinal tissue of a subject by orally administering to a subject an ingestible capsule that does not include a power source but does include at least one piezoelectric transducer for harvesting ultrasonic energy, at least one inductive coil for harvesting EM energy, an ultrasound transducer electrically coupled to said ultrasound and EM energy harvesters, and a reservoir comprising a liquid comprising a therapeutic agent or an encapsulated therapeutic agent; and transmitting via a transmitter external to the subject an EM and ultrasonic waves to the ingestible capsule to provide power for driving the ultrasound transducer to generate an ultrasound signal or ultrasound energy and thereby deliver the therapeutic agent from the reservoir into gastrointestinal tissue of the subject.

In certain aspects, methods may include administering a therapeutic agent to a gastrointestinal tissue of a subject by orally administering to a subject an ingestible capsule that does not include a power source but does include at least one tethered wire providing electrical connection to an ultrasound transducer, and a reservoir comprising a liquid comprising a therapeutic agent or encapsulated therapeutic agent; and transmitting via a transmitter external to the subject electricity to the ingestible capsule to provide power for driving the ultrasound transducer to generate an ultrasound signal and thereby deliver the therapeutic agent from the reservoir into gastrointestinal tissue of the subject. In various embodiments, the transducer comprises at least one, directional, planar, spherical, hemi-spherical, or omni-directional transducer.

In some aspects, methods can comprise administering a therapeutic agent to a gastrointestinal tissue of a subject by orally administering to a subject an ingestible capsule. In various embodiments, the therapeutic agent is encapsulated in at least one pH, thermal, electric, magnetic, electromagnetic wave, catalytic, piezo-catalytic, or ultrasound-responsive polymeric carrier, including but not limited to, microbubble, nanobubble, nanodroplet, nano emulsion, nanofiber, vesicle, micelle, or hydrogel sphere or coating. In various embodiments, the ingestible capsule comprises one or more payload or reservoir containing at least one therapeutic agent encapsulated in at least one pH, thermal, electric, magnetic, electromagnetic wave, or ultrasound-responsive polymeric carrier. In various embodiments, the ingestible capsule may comprise one or more reservoir or payload containing at least one therapeutic agent encapsulated in at least one pH, thermal, electric, magnetic, electromagnetic wave, catalytic, piezo-catalytic, or ultrasound-responsive polymeric carrier. In various embodiments, the ingestible capsule can include a coating or scaffold on at least one internal or external surface, said coating or scaffold containing at least one therapeutic agent encapsulated in at least one pH, thermal, electric, magnetic, electromagnetic wave, catalytic, piezo-catalytic, or ultrasound-responsive polymeric carrier. In various embodiments, the ingestible capsule may contain an iron oxide particle-based biocompatible gel with a controlled architecture that can release its payload containing an encapsulated or non-encapsulated therapeutic agent when exposed to at least one AC magnetic field.

Aspects of the invention may include methods of administering a therapeutic agent to a gastrointestinal tissue of a subject by transporting the ingestible capsule to at least one specific location of the GI and the payload containing an encapsulated or non-encapsulated therapeutic agent is activated by an ultrasound transducer within the capsule for control-released, pulsatile, non-pulsatile, intermittent, digital, or continuous local or targeted delivery of said agent from the payload or reservoir into gastrointestinal tissue of the subject. In various embodiments, the ingestible capsule can be swallowed by a subject or person and the payload or reservoir containing an encapsulated or non-encapsulated therapeutic agent is activated by an ultrasound transducer external to the subject, exposing ultrasound energy to the capsule, capsule payload, or capsule reservoir for control-released, pulsatile, non-pulsatile, intermittent, digital, or continuous local or targeted delivery from said reservoir or payload into gastrointestinal tissue of the subject. In various embodiments, the ultrasound transducer may be a high frequency imaging transducer used to locate, manipulate, rotate, position, or transport the capsule and to transmit ultrasonic energy to at least one of: a surface of the capsule, an energy harvesting transducer, an energy producing transducer, a payload, or a reservoir within the capsule. In various embodiments, the ultrasound transducer can be a capacitive array transducer used to focus ultrasound, locate, manipulate, rotate, position, or transport the capsule, and to transmit ultrasonic energy to at least one of: a surface of the capsule, an energy harvesting transducer, an energy producing transducer, a payload, or a reservoir within the capsule. In various embodiments, the ultrasound transducer may be a capacitive array transducer used to focus ultrasound, locate, manipulate, rotate, position, or transport the capsule, and to transmit ultrasonic energy to at least one of: a surface of the capsule, an energy harvesting transducer, an energy producing transducer, a payload, or a reservoir within the capsule. In various embodiments, the ultrasound transducer can be a high frequency phased-array transducer, used to locate, manipulate, rotate, position, or transport the capsule, and to transmit ultrasonic energy to at least one of: an internal or external surface of the capsule, an energy harvesting transducer, an energy producing transducer, a payload, or a reservoir within the capsule. In various embodiments, the ultrasound transducer may be a high frequency phased-array transducer configured to first manipulate the ingestible capsule, using non-limiting energy focusing/de-focusing or frequency sweep methods, second to deliver ultrasound energy to rupture the capsule's payload or reservoir or payload containing at least one encapsulated or non-encapsulated therapeutic agent, and third to disperse the released therapeutic agent with ultrasound energy. In some embodiments, the ingestible capsule can be transported to a specific location of the GI tract and the payload or reservoir containing an encapsulated or non-encapsulated therapeutic agent may be activated by an external or internal pH, thermal, electric, magnetic, electromagnetic wave, catalytic, or piezo-catalytic source located externally to or internally in the capsule for control-released, pulsatile, non-pulsatile, intermittent, digital, or continuous local or targeted delivery from the payload or reservoir into gastrointestinal tissue of the subject.

### BRIEF DESCRIPTION OF THE DRAWINGS

FIG. 1 illustrates an ingestible ultrasound capsule according to certain embodiments.

FIG. 2 illustrates an inductive transmitter according to certain embodiments.

FIG. 3 illustrates a wearable transmitter-receiver ultrasound-mediated drug delivery system according to certain embodiments.

FIG. 4 is a pictorial of the construction of a magnetic field power receiver 3-dimensional antenna according to certain embodiments.

FIG. 5 illustrates an ingestible ultrasound capsule comprising a source electrode according to certain embodiments.

FIG. 6 illustrates an ingestible ultrasound capsule comprising a magnetic field producer coil according to certain embodiments.

FIG. 7 is a diagram of an RF powered ingestible capsule ultrasound-mediated drug delivery system according to certain embodiments.

FIG. 8 is a diagram of an ultrasound energy powered ingestible capsule drug delivery system comprising a rectifier and a frequency multiplier according to certain embodiments.

FIG. 9 is a diagram of an ultrasound energy powered ingestible capsule drug delivery system comprising a power harvester according to certain embodiments.

FIG. 10 is a diagram of an ingestible capsule ultrasound-activated drug delivery system according to certain embodiments.

### DETAILED DESCRIPTION

### Ultrasound System and its Components

Various embodiments of the invention can provide systems and components thereof for ultrasound-mediated drug delivery within the GI tract of a subject. The systems can comprise a transmitter that includes a power source and power emitter for transmitting EM and or non-EM energy. The power transmitter may comprise one or more capacitive, inductive, magnetic resonance, RF, or ultrasonic energy radiators. A second component may include an ingestible capsule that is physically separate from the transmitter. The ingestible capsule may be configured to harvest energy from the transmitter using a variety of components. In various embodiments, the ingestible pill can comprise at least one energy harvesting component, an ultrasound transducer, and a reservoir or payload that releasably retains a liquid or powder mixture containing an encapsulated or non-encapsulated therapeutic agent. When in use, the ingestible capsule enters the gastrointestinal (GI) tract of the subject, while the transmitter remains external to the subject's body. The energy harvesting component enables miniaturization of the capsule. Therefore the capsule is small enough to be easily ingested and yet has the capacity to hold enough drug for delivering therapeutically effective doses directly to targeted tissue.

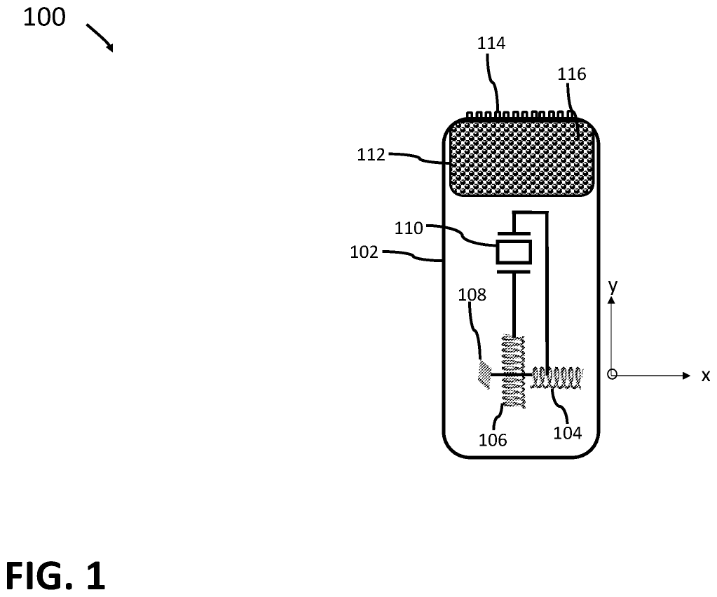

Referring now to FIG. 1, a pictorial 100 of an ingestible ultrasound capsule is shown, according to various embodiments. In various embodiments, the ingestible capsule 102 contains one or more inductive receiver coil 104, 106, 108, an ultrasound-producing transducer 110, and a reservoir 112 for holding liquid containing the therapeutic compound. In various embodiments, receiver coil 104, 106, 108 are configured with each coil's longitudinal axis at varying angle with respect to another. In a preferred embodiment, receiver coil 104, 106, 108 are configured with each coil's longitudinal axis perpendicular or orthogonal to one another to receive inductive energy independent of the capsule's orientation. For example, receiver coil 104 is configured with a longitudinal axis in a Cartesian coordinate x-direction. Receiver coil 106 is configured with a longitudinal axis in a Cartesian coordinate y-direction, and receiver coil 108 is configured with a longitudinal axis in a Cartesian coordinate z-direction. In various embodiments, receiver coil 104, 106, 108, preferably Litz wire, is wound on a ferrite core (e.g., 3F4 ferrite) to increase the power transfer. In a preferred embodiment, to maximize the received power, receiver coil 104, 106, 108 are tuned to resonate at a specific carrier frequency (e.g., 1 MHZ). Their individual contributions are added, after rectification, to avoid dephasing problems. In various embodiment, receiver coil 104, 106, 108 comprise coils with non-limiting diameter between 0.1-0.5 mm, non-limiting radius between 1-10 mm and a length between 1-10 mm. In various embodiment, receiver coil 104, 106, 108 comprise coils with a non-limiting number of windings between 20 to 50 turns, non-limiting volume between 0.25 to 1.0 cm³, non-limiting weighing 0.5-to-5.0-gram, non-limiting inductance between 10 to 100 μH, and non-limiting Q @ 1 MHz between 20 to 50. In various embodiments, reservoir 112 may contain one or more openings 114 that allow the therapeutic compound to exit reservoir 112 and enter the subject's tissue. The transducer 110 may be oriented to direct ultrasound waves toward the reservoir 112, away from the reservoir 112, radially from the capsule 102 and orthogonal to the reservoir 112, or at any angle relative to the axis between the transducer 110 and the reservoir 112. In embodiments in which ultrasound waves from the transducer 110 are directed toward the reservoir 112, the ultrasound energy facilitates transfer of a therapeutic agent from the reservoir 112 to the tissue. In embodiments in which ultrasound waves from the transducer 110 are directed away from the reservoir, an encapsulated or non-encapsulated therapeutic agent 116 may exit the reservoir 112 in proximity of the tissue by passive diffusion, and the ultrasound energy may be used to pre-treat and/or post-treat the tissue to facilitate entry of the agent 116 into the tissue.

The transducer 110 delivers ultrasound energy at a frequency optimal for promoting entry of the therapeutic agent 116 into the tissue of the GI tract. The ultrasound transducer 110 may produce an ultrasound signal of from about 10 kHz to about 10 MHz, from about 10 kHz to about 1 MHz, from about 10 kHz to about 100 kHz, from about 20 kHz to about 80 kHz, from about 20 kHz to about 60 kHz, or from about 30 kHz to about 50 kHz. The ultrasound transducer 110 may produce an ultrasound signal of less than 100 kHz, less than 80 kHz, less than 60 kHz, or less than 50 kHz. The ultrasound transducer 110 may produce an ultrasound signal of about 20 kHz, about 25 kHz, about 30 kHz, about 35 kHz, about 40 kHz, about 45 kHz, about 50 kHz, about 55 kHz, or about 60 kHz.

The design of the ingestible capsule 102 enables the transducer 110 to produce ultrasound energy at a desired power, frequency, duty cycle, or intensity. In some embodiments, the frequency of electromagnetic signal generated by the external transmitter and received by the inductive receiver 104, 106, or 108 is the same as the operating frequency of the ultrasound transducer 110. Such embodiments alleviate the need for additional circuitry within the capsule to produce an ultrasound electrical drive signal derived from a direct current (DC) power source. In other embodiments, the capsule 102 contains a component that modulates the frequency of the received electrical signal to produce the optimal transducer frequency. For example, and without limitation, a 2-diode odd-order frequency multiplier may be used to convert a 20 kHz received signal at the inductive receiver coil 104, 106, or 108 to a 60 kHz signal provided to the ultrasound drive transducer 112. Alternatively, an attenuator may be used to reduce the received frequency to achieve the desired transduction frequency. In either scenario, the modulator is placed between the inductive receiver coil 104, 106, or 108 and ultrasound transducer 112 in the electrical circuit within the capsule.

In various embodiments, the ingestible capsule 102 may have a defined size, length, or volume. For example, and without limitation, the ingestible capsule 102 may have the longest dimension of less than about 3.0 cm, about 2.75 cm, about 2.5 cm, about 2.25 cm, about 2.0 cm, about 1.75 cm, or about 1.5 cm. The ingestible capsule 102 may have a transverse dimension of less than about 1.2 cm, about 1.1 cm, about 1.0 cm, about 0.9 cm, about 0.8 cm, about 0.7 cm, about 0.6 cm, or about 0.5 cm. The ingestible capsule 102 may have a radial dimension of less than about 1.2 cm, about 1.1 cm, about 1.0 cm, about 0.9 cm, about 0.8 cm, about 0.7 cm, about 0.6 cm, or about 0.5 cm.

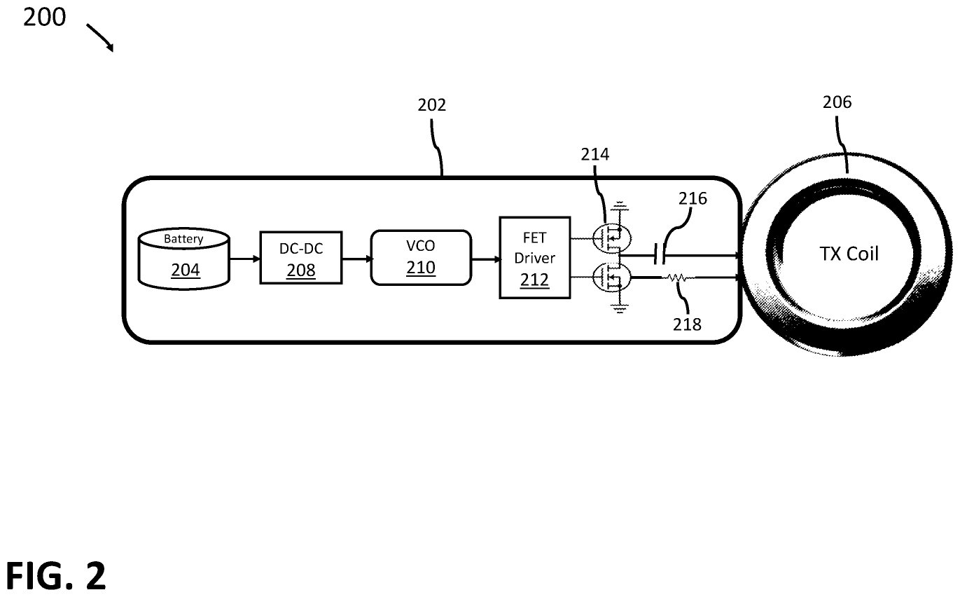

Referring now to FIG. 2, a pictorial 200 of an inductive transmitter is shown, according to various embodiments. The transmitter 202 includes a power source 204 that is electrically coupled to a transmitter coil 204. In some embodiments, the power source 204 is a battery or battery pack. The battery or battery pack may be rechargeable. The power source 204 may produce a DC voltage in a defined range. For example and without limitation, the power source may produce a DC voltage of from about 1.6 VDC to about 64 VDC, from about 3.2 VDC to about 64 VDC, from about 6.4 VDC to about 32 VDC, from about 1.6 VDC to about 32 VDC, from about 3.2 VDC to about 32 VDC, from about 6.4 VDC to about 32 VDC, from about 1.6 VDC to about 16 VDC, from about 3.2 VDC to about 16 VDC, or from about 6.4 VDC to about 16 VDC.

In various embodiments, the transmitter 202 may contain a DC-DC converter 208 between the power source 204 and the transmitter coil 206. For example, the DC-DC converter 208 may be a boost-buck DC-DC converter. The DC-DC converter 208 may increase the voltage to a defined range. For example and without limitation, the DC-DC converter 208 may increase the voltage produced by the power source 204 to from about 8 VDC to about 80 VDC, from about 16 VDC to about 160 VDC, from about 32 VDC to about 320 VDC, from about 8 VDC to about 80 VDC, from about 16 VDC to about 160 VDC, from about 32 VDC to about 320 VDC, from about 8 VDC to about 80 VDC, from about 16 VDC to about 160 VDC, or from about 32 VDC to about 320 VDC.

In various embodiments, the transmitter 202 may contain a voltage-controlled oscillator (VCO) 210 between the DC-DC converter 208 and the transmitter coil 206. The VCO 210 may generate an alternating current (AC) waveform in a defined range. For example and without limitation, the VCO 210 may generate an alternating current (AC) waveform of from about 5 kHz to about 500 kHz, from about 10 kHz to about 500 kHz, from about 20 kHz to about 500 kHz, from about 5 kHz to about 1 MHz, from about 10 kHz to about 1 MHz, from about 20 kHz to about 1 MHz, from about 5 kHz to about 2 MHz, from about 10 kHz to about 2 MHz, or from about 20 kHz to about 2 MHz.

In various embodiments, the transmitter 202 may contain a FET driver 212 and FET transistor network 214 between the VCO 210 and the transmitter coil 206. The FET driver 212 and FET transistor network 214 may switch at the frequency of the AC waveform and generate a pulsed signal at that frequency and at a voltage level equal to the output of the DC-DC converter 202.

In various embodiments, the transmitter 202 may contain a drive capacitator 216 between the FET transistor network 214 and the transmitter coil 206 and a resistor 218 electrically connected to the transmitter coil 206. In various embodiments, the value of the drive capacitor 218 and inductive windings of the transmitter coil 206 are selected such that they resonate at the same output frequency of the VCO 210 according to:

$${F}_{r} = \frac{1}{2} {π ( L * C )}^{0.5}$$

This results in the configuration of a bandpass filter that converts the pulsed output of the FET transistor network 214 to a sinusoidal waveform with sufficiently high voltage across the inductive windings of transmitter coil 206, as predicted by

$${V}_{rms} = I * j * 2 π * {F}_{r} * L$$

whereby I represent the network current established by the value of resistor 216 and L is the inductance of the inductive windings the transmitter coil 206. Thus, the drive capacitor 216 operates in conjunction with the inductive windings 206 in series resonance.

In various embodiments, the circuitry of the transmitter 202 may be adapted to produce a magnetic field or a magnetic field gradient that retains, position, or secure the ingestible capsule 102 of FIG. 1 at a specific location in the subject's body or GI tract while transmitter 202 remains external to the body. For example, output of the DC-DC converter 208 may be disconnect from the input of the FET driver 214 and connected directly to the transmitter coil 206. The use of an externally applied magnetic field to secure an ingestible ultrasound device within the body of a subject is described in International Patent Publication No. WO 2012/158648, the contents of which are incorporated herein by reference.

In various embodiments, the transmitter 202 may contain components that allow the transmitter to interact with remote devices other than the ingestible capsule 102 of FIG. 1. For example, the transmitter 202 may contain a microprocessor. The microprocess may be equipped for wireless communication with remote electronic devices, such as a computer, mobile phone, or other mobile electronic device.

In various embodiments, the transmitter 202 may contain elements that facilitate user interaction. For example, the transmitter 202 may include a user interface to receive input from a user. For example, and without limitation, the user interface may be or include a keyboard, keypad, touch screen, button, switch, knob, sensor, or the like. The transmitter 202 may include an output device that displays information to a user. For example, and without limitation, the output device may be or include a display, screen, light, or the like. The output device may display any type of information. For example, and without limitation, the output device may display information about battery charge or status of the transmitter and/or ingestible capsule.

In various embodiments, the transmitter 202 may monitor the impedance of the transmitter coil 206. A change in impedance may indicate that the transmitter 202 is in proximity to the ingestible capsule 102 of FIG. 1. Thus, the transmitter 202 may display a signal indicating that the impedance of the transmitter coil 206 has changed, thus notifying the user that transmitter 202 is close to the ingestible capsule 102 of FIG. 1. The transmitter 202 may further be programmed to energize the ingestible capsule 102 of FIG. 1 to transduce ultrasound waves and/or apply an electrical current to the reservoir 112 of FIG. 1 in response to a change, e.g., an increase or decrease, in impedance.

In various embodiments, the transmitter 202 may be configured for easy use by a person. For example, the transmitter 202 may be configured to fit in the hand of a use. In some embodiments, the transmitter 202 is generally be shaped like a wand. The transmitter 202 may include a grip or other material that facilitates physical manipulation of the device. The transmitter 202 may be configured as part of a garment that may be worn by a person. For example, and without limitation, the transmitter may be integrated into a glove, vest, shirt, jacket, belt, piece of headgear, goggles, or another wearable item. In an alternative embodiment, transmitter coil 206 comprises a solenoid coil that can wrap around a subject's chest, stomach, or trunk. The transmitter 202 may be configured to connect to an external power source that recharges the internal battery 204.

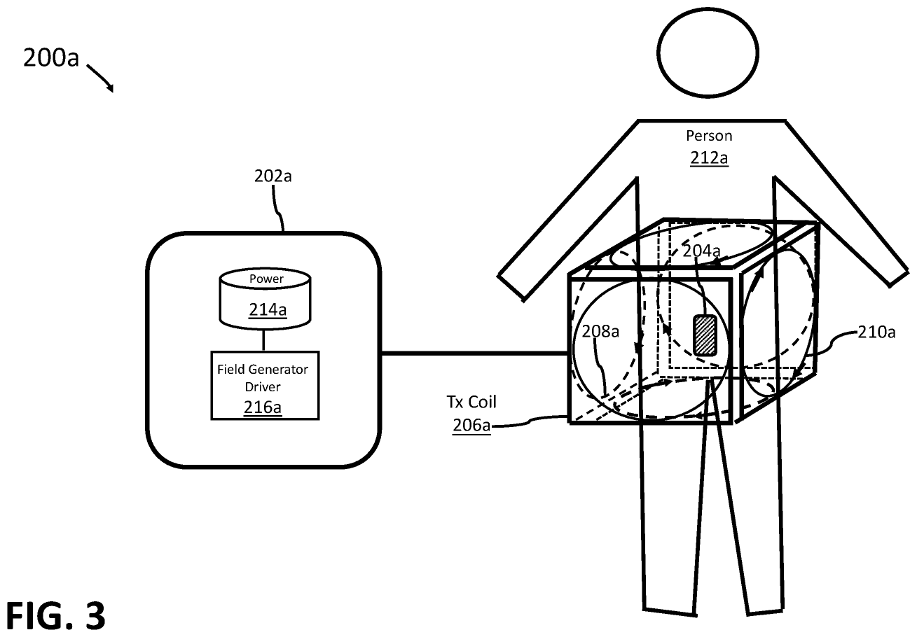

Referring now to FIG. 3, a pictorial 200a of a wearable transmitter-receiver ultrasound-mediated drug delivery system is shown, according to various embodiments. The drug delivery system can comprise a wearable transmitter 202a operating in conjunction with an ingestible capsule 204a. In various embodiments, the transmitter 202a operates in combination with at least one Helmholtz coil 206a field generator, preferable a coil configured to transmit and expose one or more magnetic field or flux generated by one or more coil 208a, 210a, current flows drawn as dashed elliptic-circular arrowed lines, to ingestible capsule 204a for energy transfer to the capsule ingested by a person 212a. In a preferred embodiment, ingestible capsule 204a is equivalent to capsule 102 of FIG. 1. In an alternative embodiment, capsule 204a comprises or lacks at least one additional component to operate in combination with transmitter 202a in various configurations. In a preferred embodiment, Helmholtz coil 206a comprises at least one wearable square, circular, cylindrical, or cubic coil and configured to be worn around person 212a's chest, torso, or trunk. In an alternative embodiment, Helmholtz coil 206a is configured within a chamber or an electromechanical structure that enables person 212a's body chest, torso, or trunk to be exposed to expose one or more magnetic field or flux generated by one or more coil 208a, 210a to provide energy transfer to ingestible capsule 204a. In yet another alternative embodiment, Helmholtz coil 206a is configured as a solenoid capable of surrounding a person 212a to enable person 212a's body chest, torso, or trunk to be exposed to one or more magnetic field or flux generated by one or more coil 208a, 210a thus providing energy transfer to ingestible capsule 204a. In various embodiments, ingestible capsule 204a can be exposed to one or more magnetic field or flux in an arbitrary position and or orientation within person 212a's GI tract. In various embodiment, three pairs of coils are arranged a cubic formation to generated three different magnetic fields, with coils opposite to each other forming a pair and carry current in phase, to produce a homogeneous magnetic field orthogonal to their planes. In one embodiment, transmitter coil 206a comprises square coils and in alternative embodiment, transmitter coil 206a comprises circular coils. In a preferred embodiment, the coils comprise rectangular or cylindrical Litz wires to reduce resistive losses at high frequency. The Litz wire may comprise a wire with non-limiting wire diameter between 0.1 mm to 2.00 mm. In various embodiments, transmitter 202a can comprise a power source 214a, for example battery 204 of FIG. 2, electrically connected to power at least one electrical component of transmitter 202a, including a field generator driver 216a. In various embodiment, field generator driver 216a may comprise an inverter to generate one or more sinusoidal high-amplitude electric current through at least one coil, for example a solenoid coil of Helmholtz coil 206a. In a preferred embodiment, the inverter is a Class E inverter. In various embodiments, one or more component of said Class E inverter can be exchanged to change its operation. For example, a parallel capacitance can be replaced with a diode to reduce the inverter to insensitive to variations in one or more resistors of the Class E inverter. Without limitation, the inverter may generate an alternating current (AC) waveform of from about 5 kHz to about 500 kHz, from about 10 kHz to about 500 kHz, from about 20 kHz to about 500 kHz, from about 5 kHz to about 1 MHz, from about 10 kHz to about 1 MHz, from about 20 kHz to about 1 MHZ, from about 5 kHz to about 2 MHz, from about 10 kHz to about 2 MHz, or from about 20 kHz to about 2 MHz.

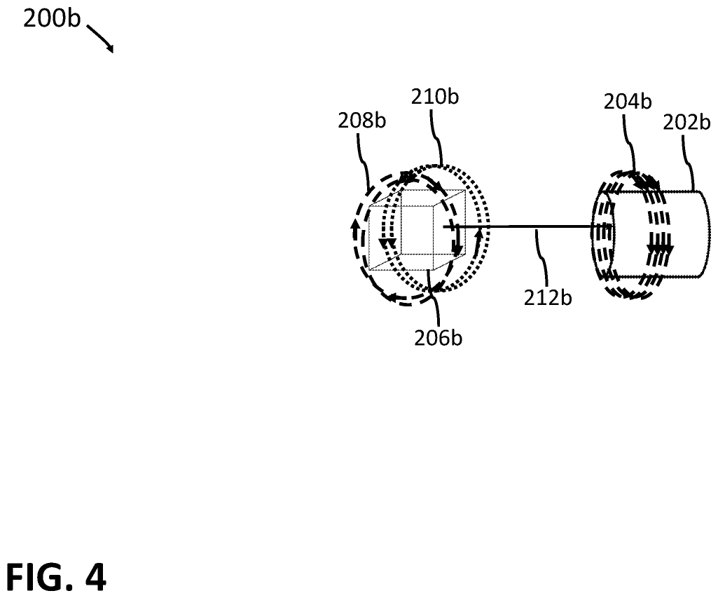

Referring now to FIG. 4, a pictorial 200b of the construction of a magnetic field power receiver 3-dimensional antenna is shown, according to various embodiments. The magnetic field power receiver antenna can comprise three orthogonally directed coils and integrated into the ingestible capsule 102 of FIG. 1. In various embodiments, the magnetic field power receiver antenna may operate in conjunction with one or more transmitter coil 206a of FIG. 3 or at least one solenoid coil. In various embodiments, the three orthogonal coils are configured and fabricated with the construction of an outer cylindrical coil and two inner orthogonal square coils. First, one or more winding electrical wire is wound or wrapped cylindrically around a cylinder scaffold 202b to create an outer cylindrical coil 204b. Second, one or more winding electrical wire is wound or wrapped around a square scaffold 206b to create coil 208b. Third, one or more winding electrical wire is wrapped on top and perpendicular to coil 208b that has been wrapped around square scaffold 206b to create coil 210b. In various embodiments, the square scaffold 206b may be constructed from a diamagnetic, paramagnetic, magnetic, or ferromagnetic material to create a frame, hollow cage, or solid cube. In a final step, cylinder scaffold 202b is removed from outer cylindrical coil 204b and the square scaffold 206b containing coil 208b and 210b is inserted, shown by arrow 212b into outer cylindrical coil 204b to form a 3-dimensional magnetic field power receiver antenna. The resulting antenna comprises cylindrical coil 204b that is sensitive in the Cartesian coordinate x-axis, coil 208b that is sensitive in the Cartesian coordinate y-axis, and coil 206b that is sensitive in the Cartesian coordinate z-axis. The resulting antenna and each coil are electrically connected to a power receiver circuit network. In various embodiments, cylindrical coil 204b may have a length creating an internal volume that accommodate coil 206b and coil 208b and a power receiver electronic network within the coil's core for driving one or more loads including ultrasound transducer 110 of FIG. 1. In various embodiments, the power receiver electronic network can comprise parallel rectifiers, with at least one operational due to the orthogonality of the receiver coils 204b, 206b, 208b. In various embodiments, receiver electronic network may comprise one or more regulators to power on or more additional components, for example, sensor, data transmitter, or miniature camera.

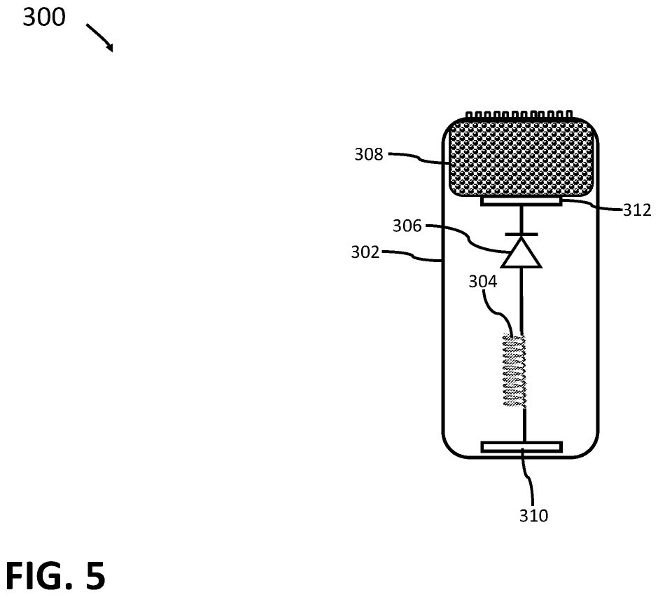

Referring now to FIG. 5, a pictorial 300 of an ingestible ultrasound capsule is shown, according to various embodiments. The ingestible capsule 302 contains an inductive receiver coil 304, a rectifier 306, a reservoir 308, a return electrode 310, and a source electrode 312. In this embodiment, the capsule 302 is equipped to receive an AC signal at the inductive receiver coil 304 and rectify it at the rectifier 306 to produce a DC signal or DC pulse train. In various embodiments, without limitation, the rectifier 306 may be a single diode to produce pulses, or it may be a full wave bridge rectifier to establish a DC level. The DC signal or DC pulse train is then provided to the source electrode 312, which is electrically connected to the reservoir 308, equivalent to reservoir 112 of FIG. 1. In certain embodiments, by charging the encapsulated or non-encapsulated therapeutic compound or providing it in a conductive medium, the energized source electrode 312 drives the therapeutic compound into the surrounding GI tissue by iontophoresis. The return electrode 310 contacts the GI tissue to complete the electrical circuit required for iontophoresis. In other embodiments, higher energy pulses are substituted for iontophoresis to drive the therapeutic compound into the surrounding GI tissue by electroporation. In other embodiments, iontophoresis or electroporation electrical signal is directed away from the reservoir 308 and applied directly to the surrounding GI tissue to pretreat or post-treat tissue while the therapeutic compound is allowed to passively diffuse out of the reservoir 308 into the surrounding GI tissue.

The ingestible capsule 302 may also contain other electronic components. For example, and without limitation, the ingestible capsule 302 may contain one or more of a video cameras, components for management of the camera, and components for communications between the ingestible capsule 302 and external devices. Images obtained from a video camera may be used to identify GI ulcerations or regions of inflammation. In some embodiments, the ingestible capsule 302 contains a microprocessor with Bluetooth capability to capture video images and communicate with an external mobile device. In some embodiments, the ingestible capsule 302 contains a pH sensor and a microprocessor that manages the pH sensor and communicates local pH measurements within the GI tract to an external mobile device. Because the pH varies throughout the GI tract, pH measurements may be used to identify the anatomical location of the ingestible capsule 302 at a given point in time. Based on information obtained from video images, pH measurements, or both, the ingestible capsule may be selectively energized for ultrasound transduction and/or electrode-driven iontophoresis or electroporation at specific locations within the GI tract. Consequently, the system enables targeted delivery of therapeutic compounds to achieve the optimal therapeutic benefit.

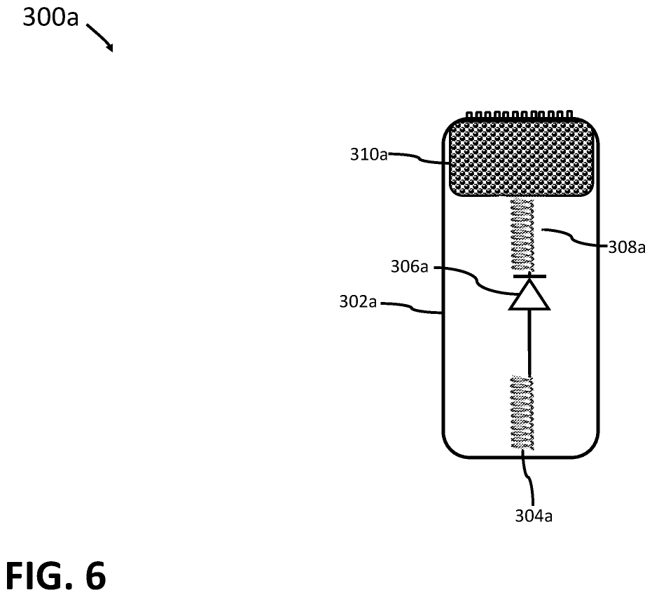

Referring now to FIG. 6, a pictorial 300a of an ingestible ultrasound capsule is shown, according to various embodiments. The ingestible capsule 302a contains at least one inductive receiver coil 304a, a rectifier 306a, a magnetic field producer coil 308a, and reservoir 310a. In this embodiment, the capsule 302a is equipped to receive an AC signal at the inductive receiver coil 304a and rectify it at the rectifier 306a to produce a DC signal or DC pulse train. In a preferred embodiment, ingestible capsule 302a comprises magnetic field power receiver 3-dimensional antenna described in FIG. 4. In various embodiments, without limitation, the rectifier 306a may be a single diode to produce pulses, or it may be a half-wave or full wave bridge rectifier to establish a DC level or AC level. In various embodiments, one or more AC or DC signal is provided to magnetic field producer coil 308a which exposes one or more magnetic field or flux to the reservoir 310a. In certain embodiments, a transient pulse or alternating magnetic pulse or flux is exposed to the reservoir which is configured to store a liquid, a mixture, a matrix, or scaffold containing at least one therapeutic agent and a magnetically responsive fluid or polymeric carrier, including but not limited to, microbubble, nanobubble, nanodroplet, nano emulsion, nanofiber, vesicle, micelle, or hydrogel sphere or coating. In various embodiments, said pulsed or alternating magnetic field release said therapeutic agent from the reservoir. In one embodiment, said fluid or polymeric carrier comprises a diamagnetic, paramagnetic, superparamagnetic, or ferromagnetic nano or microparticle. In various embodiments, the nano or microparticle may comprise Fe₂O₃, Fe₃O₄, or BaTiO₃ that may be modified to releasably bind with at least one said therapeutic agent. In an alternative embodiment, an external pulsed or alternating magnetic field may be used to release said therapeutic agent from said reservoir 310a configured with said magnetically responsive fluid or polymeric carrier. In this embodiment, one or more external field is generated using, for example, transmitter 202a of FIG. 3, via transmitter coil 206a. In one embodiment, ingestible capsule 204a of FIG. 3 can be configured in similar manner to incorporate said magnetically responsive fluid or polymeric carrier and a therapeutic agent within its reservoir. In yet another alternative embodiment, the external field generated by transmitter 202a of FIG. 3 transport previously released said therapeutic into the surrounding tissue of the GI tract.

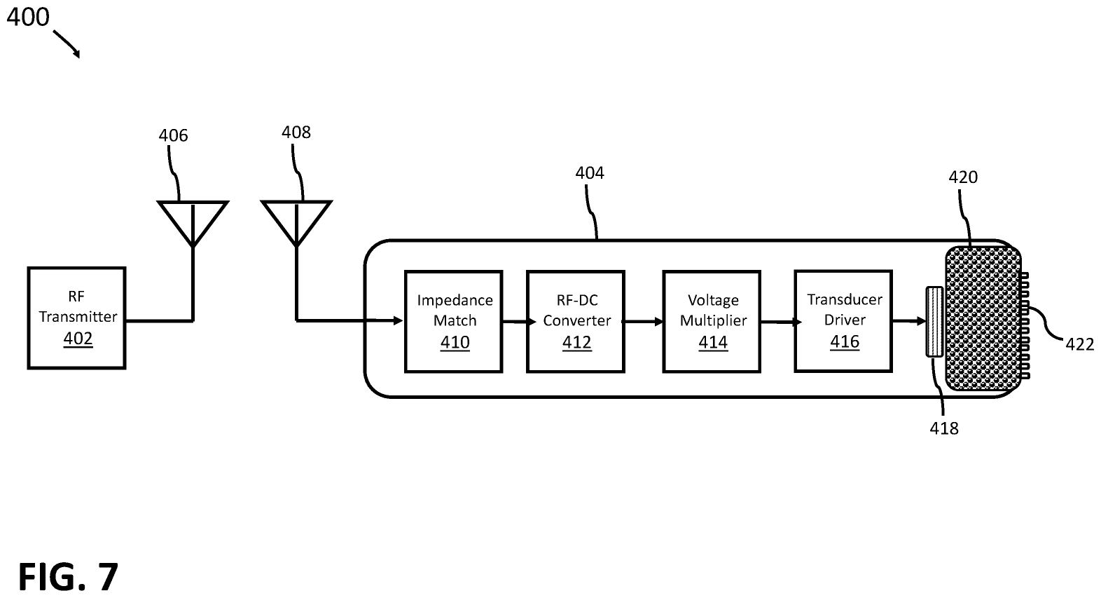

Referring now to FIG. 7, a diagram 400 of an RF powered ingestible capsule ultrasound-mediated drug delivery system is shown, according to various embodiments. The ingestible capsule ultrasound-mediated drug delivery system comprises a radiofrequency (RF) energy transmitter 402 configured to operate in conjunction an ingestible capsule 404. In various embodiments, energy transmitter 402 comprises a RF generator-transmitter to broadcast RF energy from antenna 406. In various embodiments, ingestible capsule 404 may comprise at least one RF energy or signal receiving antenna 408, an impedance matching network 410, an RF-DC converter 412, voltage multiplier 414, an ultrasound transducer driver 416, ultrasound transducer 418, and reservoir 420. In various embodiments, ultrasound transducer 418 is configured to irradiate reservoir 420 with ultrasound energy to transport a drug payload out of said reservoir 420 to through one or more orifice 422. In various embodiments, RF transmitter 402 comprises an energy storage device, microcontroller, power management module, and RF transceiver. RF transmitter 402, preferable a portable device, operates external to the body and GI tract of a person who has ingested capsule 404. Without limitation, RF transmitter 402 the may generate RF energy or EM wave broadcast by antenna 406 in the range of about 5 kHz to about 500 kHz, from about 10 kHz to about 500 kHz, from about 20 kHz to about 500 kHz, from about 5 kHz to about 1 MHz, from about 10 kHz to about 1 MHz, from about 20 kHz to about 1 MHz, from about 5 kHz to about 2 MHz, from about 10 kHz to about 2 MHz, from about 20 kHz to about 2 MHz, from about 2 MHz to 200 MHz, from 200M to 2 GHz, or from 2 GHz to 10 GHz. In various embodiments, antenna 406 comprises an isotropic antenna or directional antenna.

The ingestible capsule 404 of the ultrasound-mediated drug delivery system the receives RF energy or wave transmitted by RF transmitter 402 with antenna 408. In various embodiments, antenna 408 is configured with non-limiting shape, size, and dimensions, for efficient reception of RF energy at an optimal form factor to minimize or reduce the size or volume of ingestible capsule 404. In various embodiments, antenna 408 can be miniaturized by modifying one or more basic patch shapes and embedding one or more suitable slots in the radiating patch. In various embodiments, antenna 408 comprises one or more antenna shape, including but not limited to, circular patch with slots place on the diameter, square patch with cross-shaped slot etched on its surface, slits on the perimeter of a square patch, square patch with two orthogonal pairs of regular or irregular, symmetrical or non-symmetrical slits, the like, or combination thereof. In various embodiments, antenna 408 may be fabricated using various substrates, including but not limited to, FR-4 substrate, Arlon substrates, Tarconic, TLY-5 laminate, RT/Duroid 6010 substrate, RT/Duroid 5870 substrate. In various embodiments, antenna 408 is configured with one or more slots to increase its electrical path to lengthen the surface current paths as a miniature antenna. In various embodiments, antenna 408 comprises a cylindrical or rectangular dielectric resonator antenna (DRA) for operation at high frequencies.

The RF energy received by antenna 408 of ingestible capsule 404 is fed into an impedance matching network 410 to reduce the transmission loss from the antenna to RF-DC converter 412 or rectifier. In various embodiments, matching network 410 comprises on or more reactive components, non-dissipative coil, and capacitor. In various embodiments, matching network 410 comprises a transformer, shunt inductor, or LC network. Matching network 410 for RF energy harvesting may comprise, but not limited to, L-type, π-type, and T-type matching networks. The impedance matching network 410 functions to maximize the energy or power transfer from the receiving antenna 408 to the RF-D rectifier 410 circuit and increases the RF input voltage level for the rectifier.

The ingestible capsule 404 of the ultrasound-mediated drug delivery system contains an RF-DC converter as the main block for the RF energy harvesting system. In various embodiments, RF-DC converter 412 or rectifier converts RF power captured by antenna 408 into useable DC power. In various embodiments, RF-DC converter 412 may comprises, but not limited to, a diode-base, bridge of diode, or voltage multiplier. In one embodiment, the topology for the rectifier circuit of RF-DC converter 412 is a full-wave rectifier. The full-wave rectifier converts both half-cycles (positive half-cycle and negative half-cycle) of the RF signal into a pulsating DC signal. The RF-DC converter 412 may operate in conjunction with one or more voltage multiplier.

The ingestible capsule 404 of the ultrasound-mediated drug delivery system contains a voltage multiplier. In various embodiments, voltage multiplier 414 comprises one or more cascade rectifier unit that generates an output voltage higher than the input voltage. In various embodiments, voltage multiplier 414 may comprise, but not limited to, one or more Cockcroft-Walton, Greinacher, Dickson, or Villard multiplier. In various embodiments, voltage multiplier 414 may be configured to provide a specific power input for transducer driver 416.

The ingestible capsule 404 of the ultrasound-mediated drug delivery system contains a transducer driver 416 to activate an ultrasonic transducer 418 for dispersing one or more encapsulated or non-encapsulated therapeutic agent from a payload or reservoir 420. In various embodiments, transducer driver 416 may comprise, but not limited to, a shunt-C class E-amplifier to drive transducer 418. In one embodiment, the amplifier contains a parallel inductor that can resonate at for example, 40 kHz, with the transducer, fabricated with Lead Zirconate Titanate (PZT). In various embodiments, one or more series capacitors is used to prevent DC feed-through. In various embodiments, one or more series inductor is used to improve amplifier efficiency. In various embodiments, transducer driver 416 may comprise, but not limited to one more microcontroller operating in conjunction with one or more NMOS transistors to activate the input supply voltage across the PZT transducer 418. For example, a 60 kHz, pulse width modulated with a chosen duty cycle drives the gates one or more transistors to switch a supply voltage across the activating PZT transducer to propel a therapeutic agent through one or more orifice 422.

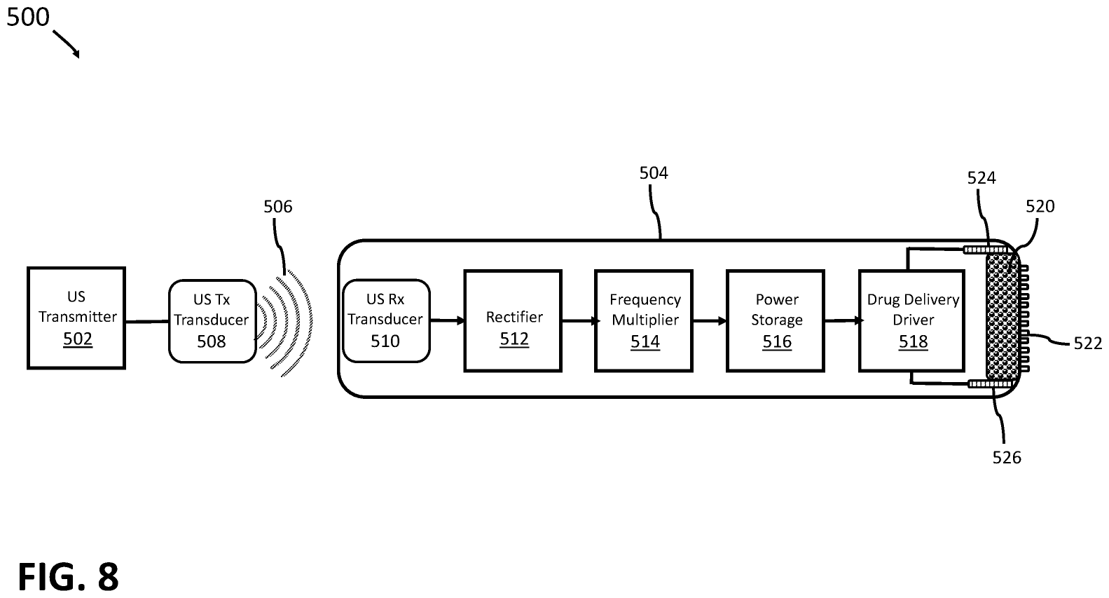

Referring now to FIG. 8, a diagram 500 of an ultrasound energy powered ingestible capsule drug delivery system is shown, according to various embodiments. The ingestible capsule drug delivery system comprises an ultrasound (US) energy transmitter 502 configured to operate in conjunction an ingestible capsule 504. In various embodiments, energy transmitter 502 comprises a US generator-transmitter to broadcast US energy or US wave 506 from US transducer 508. In various embodiments, ingestible capsule 504 may comprise at least one US receiving transducer 510, a rectifier network 512, frequency multiplier 514, power storage device 516, a drug delivery driver 518, and reservoir 520 containing at least one therapeutic agent. In various embodiments, drug delivery driver 518 is configured to transport a drug payload or a therapeutic from reservoir 520 and externally to the capsule through one or more orifice 522 thus exposing or delivering said therapeutic agent to GI tissue. In various embodiments, drug delivery driver 518 may be configured to dispense, release, or transport said therapeutic agent from reservoir 520 using iontophoresis. In one embodiment, drug delivery driver 518 comprises a two-electrode system connected to reservoir 520. The two-electrode system contains a carbon working electrode 524 and an Ag/AgCl counter electrode 526. In various embodiments, counter electrode 526 may operate as a share counter/reference electrode for the system. In various embodiments, the release or transport of said therapeutic agent from reservoir 520 is caused by oxidation/reduction, ion transport, or pH change of a liquid, a mixture, a matrix, polymer, scaffold containing the therapeutic agent and an electromagnetically responsive fluid or polymeric carrier, including but not limited to, microbubble, nanobubble, nanodroplet, nano emulsion, nanofiber, vesicle, micelle, or hydrogel sphere or coating, the like, or combination thereof.

In various embodiments, US transmitter 502 comprises an energy storage device, microcontroller, and power management module. US transmitter 502, preferable a portable device, operates external to the body and GI tract of a person who has ingested capsule 504. Without limitation, US transmitter 502 the may generate US energy or mechanical wave broadcast by US transmitting transducer 508 in the range of about 5 kHz to about 500 kHz, from about 10 kHz to about 500 kHz, from about 20 kHz to about 500 kHz, from about 5 kHz to about 1 MHz, from about 10 kHz to about 1 MHz, from about 20 kHz to about 1 MHz, from about 5 kHz to about 2 MHz, from about 10 kHz to about 2 MHz, from about 20 kHz to about 2 MHz, from about 2 MHz to 200 MHz, from 200M to 2 GHz, or from 2 GHz to 10 GHz. In various embodiments, US transmitting transducer 508 comprises at least one piezoelectric transducer, two or more piezoelectric transducers, or an array of piezoelectric electric transducers. In various embodiments, one or more piezoelectric transducer of US transmitting transducer 508 may be configured to produce and transmit pulsed or alternating diffuse, directional, omnidirectional, spherical, or focused ultrasound energy or wave to ingestible capsule 504. In various embodiments, US transmitting transducer 508 delivers ultrasound energy to US receiving transducer 510, reservoir 520, an external proximity of ingestible capsule 504, GI tissue, GI tract, said therapeutic that has been transported from reservoir 520, or combination thereof. In various embodiments, US transmitting transducer 508 may be configured or designed to have a large focusing gain, confined beam, operate at frequencies, or larger apertures for a given transmission depth to deliver energy to US receiving transducer 510.

The ingestible capsule 504 of the ultrasound energy powered ingestible capsule drug delivery system the receives US energy or mechanical wave transmitted by US transmitter 502 with US receiver transducer 510. In various embodiments, US receiver transducer 510 is configured or designed with non-limiting shape, size, dimensions, backing material, electrode, or air backing for efficient reception of US energy at an optimal form factor to minimize or reduce the size or volume of ingestible capsule 504. In various embodiments, US receiver transducer 510 may be configured to operate in one or more non-limiting mode, for example radial, flexural, planar, transverse, or longitudinal thickness mode. In a preferred embodiment, US receiver transducer 510 designed to simultaneously achieve small dimensions with optimal impedance for efficient energy harvesting, thus resulting in a miniature form factor or size for ingestible capsule 504. In various embodiments, US receiver transducer 510 may be constructed with millimeter dimensions or scale to minimize parasitic modes, achieve large reception acceptance angle, reduce the impact of power losses due to capsule orientation, and reduce tissue transmission losses at long depths using high operating frequencies. In various embodiments, the ultrasound energy powered ingestible capsule drug delivery system may configured for US receiver transducer 510 to operate on resonant or off-resonance, between its short and open circuit resonance or inductive band, with US transmitter 502 broadcasting with non-limiting frequencies between 0.75 to 2 MHz.

The US energy harvested by US receiver transducer 510 of ingestible capsule 404 is in the form of AC power which is the converted into DC energy via one or more power recovery network circuits. In various embodiments, the power recovery network may incorporate an impedance matching network, for example matching network 410 of FIG. 7 to reduce the transmission loss. The impedance matching network functions to maximize the energy or power transfer from the US receiver transducer 510 a rectifier 512 circuit. In various embodiments, the rectifier converts US power harvested into useable DC power. In various embodiments, rectifier 512 may comprise, but not limited to, a diode-base, bridge of diode, or voltage multiplier. In one embodiment, the topology for the rectifier circuit is a full-wave rectifier. The full-wave rectifier converts both half-cycles (positive half-cycle and negative half-cycle) of the RF signal into a pulsating DC signal. The rectifier 512 may operate in conjunction with one or more voltage multiplier. In various embodiments, a low dropout regulator may be incorporated to provide on or more DC rail for various auxiliary components such as oscillator, clocks, microcontrollers, or the like.

The ingestible capsule 504 of the ultrasound energy powered ingestible capsule drug delivery system contains a high frequency voltage multiplier. In various embodiments, voltage multiplier 514 comprises one or more cascade rectifier unit that generates an output voltage higher than the input voltage. In various embodiments, voltage multiplier 514 may comprise, but not limited to, one or more Cockcroft-Walton, Greinacher, Dickson, or Villard multiplier. In various embodiments, voltage multiplier 514 may be configured to provide a specific power input for drug delivery driver 418. In various embodiments, the power is stored within a power storage unit 516 such as a capacitor or supercapacitor.

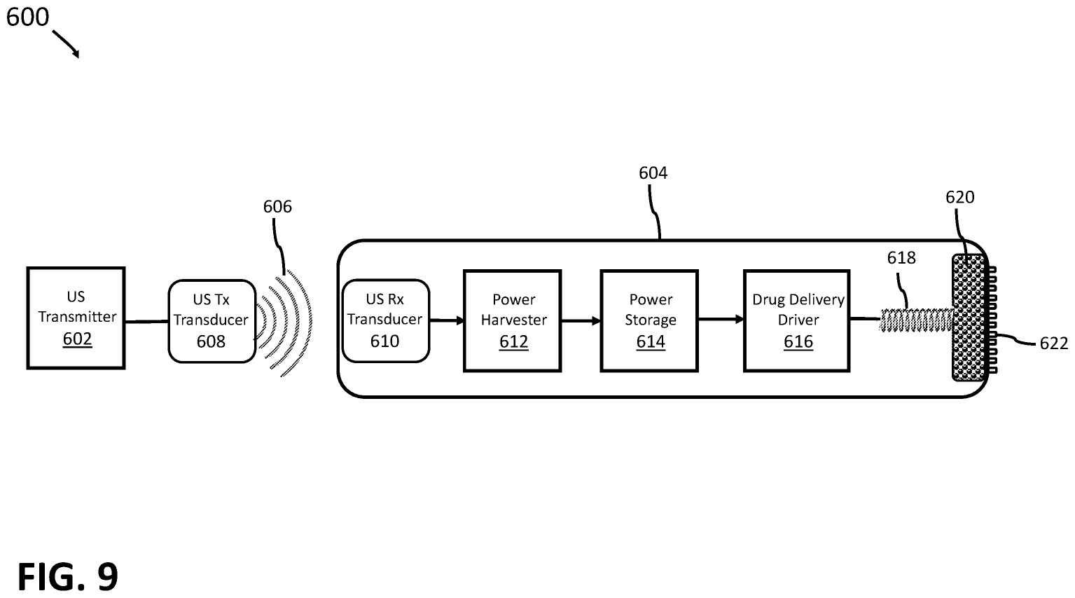

Referring now to FIG. 9, a diagram 600 of an ultrasound energy powered ingestible capsule drug delivery system is shown, according to various embodiments. The ingestible capsule drug delivery system comprises an ultrasound (US) energy transmitter 602 configured to operate in conjunction an ingestible capsule 604. In various embodiments, energy transmitter 602 comprises a US generator-transmitter to broadcast US energy or US wave 606 from US transducer 608. In various embodiments, ingestible capsule 604 may comprise at least one US receiving transducer 610, a power harvester/recovery network 612, a power storage unit 614, drug delivery driver 616, and one or more inductive coil 618, and reservoir 620 containing at least one therapeutic agent. In various embodiments, network 612 may comprise one or more rectifier network and frequency multiplier, for example rectifier network 512 and frequency multiplier 514 of FIG. 8. In various embodiments, drug delivery driver 618 is configured to transport a drug payload or a therapeutic from reservoir 620 and externally to the capsule to through one or more orifice 622 thus exposing or delivering said therapeutic agent to GI tissue. In various embodiments, drug delivery driver 616 may be configured to dispense, release, or transport said therapeutic agent from reservoir 620 by activating at least one inductive coil 618. In various embodiments, the release or transport of said therapeutic agent from reservoir 620 is caused by at least one pulse or alternating magnetic field, flux, gradient, or force applied to a liquid, a mixture, a matrix, polymer, scaffold containing the therapeutic agent and an electromagnetically, preferably magnetic responsive fluid or polymeric carrier, including but not limited to, microbubble, nanobubble, nanodroplet, nano emulsion, nanofiber, vesicle, micelle, or hydrogel sphere or coating, located with reservoir 620. In various embodiments, said pulsed or alternating magnetic field, flux, gradient, or force release said therapeutic agent from the reservoir. In one embodiment, said fluid or polymeric carrier comprises a diamagnetic, paramagnetic, superparamagnetic, magnetic, or ferromagnetic nano or microparticle. In various embodiments, the nano or microparticle comprises FezO₃, Fe₃O₄, or BaTiO₃ that may be modified to releasably bind with at least one said therapeutic agent. In an alternative embodiment, an external pulsed or alternating magnetic field may be used to release said therapeutic agent from said reservoir 620 configured with said magnetically responsive fluid or polymeric carrier. In this embodiment, one or more external field is generated using, for example, transmitter 202a of FIG. 3, via transmitter coil 206a.

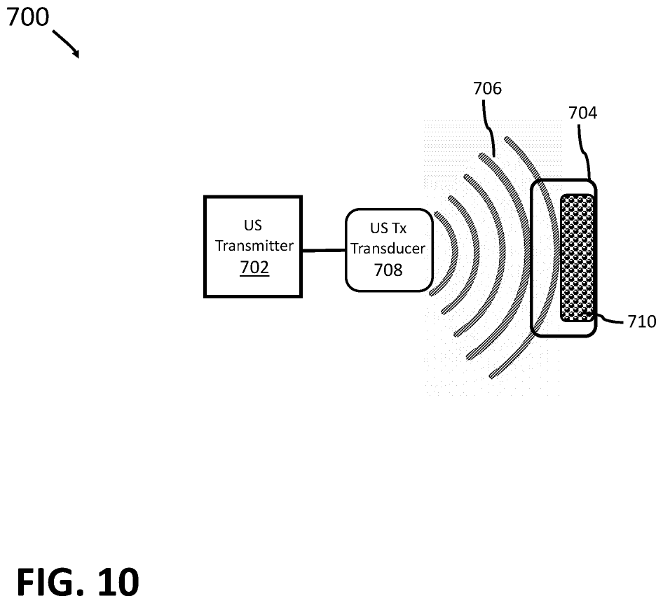

Referring now to FIG. 10, a diagram 700 of an ingestible capsule ultrasound-activated drug delivery system is shown, according to various embodiments. The ingestible capsule ultrasound-activated drug delivery system comprises an ultrasound (US) energy transmitter 702 configured to operate in conjunction an ingestible capsule 704. In various embodiments, energy transmitter 702 comprises a US generator-transmitter to broadcast US energy or US wave 706 from US transducer 708. In various embodiments, ingestible capsule 704 may comprise at least one reservoir 710 at least one liquid, a mixture, a matrix, polymer, or scaffold containing at least one therapeutic agent. In various embodiments, energy transmitter 702 and from US transducer 708 designed and configured to transmit, irradiate, or expose ultrasound energy to ingestible capsule 704 located within a person's body, preferably within one more GI tract. In various embodiments, US energy exposure to ingestible capsule 704 causes the dispersion, release, or transport of said therapeutic agent from within reservoir 710 and externally into the GI tract or one or more GI tissue. In various embodiments, the release or transport of said therapeutic agent from reservoir 710 is caused by at least one pulse or alternating ultrasonic field, flux, gradient, or force, streaming force, or cavitation force applied to said liquid, mixture, or matrix, polymer, scaffold containing the therapeutic agent and an electromagnetically, preferably magnetic responsive fluid or polymeric carrier, including but not limited to, microbubble, nanobubble, nanodroplet, nano emulsion, nanofiber, vesicle, micelle, or hydrogel sphere or coating, located with reservoir 710. Without limitation, US transmitter 702 the may generate US energy or mechanical wave broadcast by US transmitting transducer 708 in the range of about 5 kHz to about 500 kHz, from about 10 kHz to about 500 kHz, from about 20 kHz to about 500 kHz, from about 5 kHz to about 1 MHz, from about 10 kHz to about 1 MHz, from about 20 kHz to about 1 MHz, from about 5 kHz to about 2 MHz, from about 10 kHz to about 2 MHZ, from about 20 kHz to about 2 MHz, from about 2 MHz to 200 MHz, from 200M to 2 GHz, or from 2 GHz to 10 GHz. In various embodiments, US transmitting transducer 708 comprises at least one piezoelectric transducer, two or more piezoelectric transducers, or an array of piezoelectric electric transducers. In various embodiments, one or more piezoelectric transducer of US transmitting transducer 708 may be configured to produce and transmit pulsed or alternating diffuse, directional, omnidirectional, spherical, or focused ultrasound energy or wave to ingestible capsule 704. In various embodiments, US transmitting transducer 708 delivers ultrasound energy reservoir 710, an external proximity of ingestible capsule 704, GI tissue, GI tract, said therapeutic that has been transported or dispersed from reservoir 720, or combination thereof. In various embodiments, US transmitting transducer 708 may be configured or designed to have a large focusing gain, confined beam, operate at frequencies, or larger apertures for a given transmission depth to deliver energy to ingestible capsule 704. In various embodiments, US transmitting transducer 708 may be configured or designed to transport the ingestible capsule 704 to at least one specific location of the GI whereby a payload within reservoir 702 containing an encapsulated or non-encapsulated therapeutic agent is activated by ultrasound transducer 708 to disperse from the capsule for control-released, pulsatile, non-pulsatile, intermittent, digital, or continuous local or targeted delivery of said agent into gastrointestinal tissue of a person. In various embodiments, the ingestible capsule 704 is swallowed by said person and the payload or reservoir containing an encapsulated or non-encapsulated therapeutic agent is activated by ultrasound transducer 708 external to the subject, exposing ultrasound energy to the capsule 704, capsule payload, or capsule reservoir 710 for control-released, pulsatile, non-pulsatile, intermittent, digital, or continuous local or targeted delivery from said reservoir or payload into gastrointestinal tissue of said person In various embodiments, the ultrasound transducer 708 is a high frequency imaging transducer, use to locate, manipulate, rotate, position, or transport the capsule 704, and to transmit ultrasonic energy to at least one surface of the capsule. In various embodiments, the ultrasound transducer 704 is a capacitive array transducer, use to focus ultrasound, locate, manipulate, rotate, position, or transport the capsule, and to transmit ultrasonic energy to at least one; surface of the capsule, payload, or reservoir within the capsule 704. In various embodiments, the ultrasound transducer 708 is a capacitive array transducer, use to focus ultrasound, locate, manipulate, rotate, position, or transport the capsule, and to transmit ultrasonic energy to at least one; internal or external surface of said capsule, payload, or reservoir within the capsule. In various embodiments, the ultrasound transducer 708 is a high frequency phased-array transducer, use to locate, manipulate, rotate, position, or transport the capsule 704, and to transmit ultrasonic energy to at least one; internal or external surface of said capsule, payload, or reservoir within the capsule. In various embodiments, the ultrasound transducer 704 is a high frequency phased-array transducer configured to first manipulate the ingestible capsule 704, using non-limiting energy focusing/de-focusing or frequency sweep methods, second to deliver ultrasound energy to rupture said capsule's payload or reservoir 710 or payload containing at least one encapsulated or non-encapsulated therapeutic agent, and third to disperse the released therapeutic agent with ultrasound energy. In alternative embodiments, the ingestible capsule 704 is transported to a specific location of the GI tract and the payload or reservoir 710 containing an encapsulated or non-encapsulated therapeutic agent is activated by external ultrasound transducer 708 for control-released, pulsatile, non-pulsatile, intermittent, digital, or continuous local or targeted delivery from the payload or reservoir 710 into gastrointestinal tissue of said person.

An object of the present disclosure is the encapsulation of therapeutic agents with a liquid, mixture, scaffold, or responsive polymer for incorporation into a reservoir of an ingestible capsule, for example ingestible capsule 102 of FIG. 1, ingestible capsule 204a of FIG. 2, ingestible capsule 302 of FIG. 5, ingestible capsule 302a of FIG. 6, ingestible capsule of 404 of FIG. 7, ingestible capsule 504 of FIG. 8, ingestible capsule 604 of FIG. 9, or ingestible capsule 704 of FIG. 10. In various embodiments, the therapeutic agent is encapsulated in at least one pH, thermal, electric, magnetic, electromagnetic wave, catalytic, piezo-catalytic, or ultrasound-responsive polymeric carrier, including but not limited to, microbubble, nanobubble, nanodroplet, nano emulsion, nanofiber, vesicle, micelle, or hydrogel sphere or coating. In various embodiments, microbubble, nanobubble, nanodroplet, nano emulsion, nanofiber, vesicle, micelle, or hydrogel sphere or coating of the present disclosure may be produced from, but not limited to, poly(lactic acid), poly(allylamine hydrochloride), perfluorocarbon, polyvinyl alcohol, poly(lactic-co-glycolic acid, perfluoroctanol-poly(lactic acid). In various embodiments, pH or ultrasound-responsive polymer may comprise a scaffold, gel, or vesicle produce from, but not limited to, self-assembled from a poly(ethylene oxide)-block-poly[2-(diethylamino)ethyl methacrylate-stat-2-tetrahydrofuranyloxy) ethyl methacrylate] [PEO-b-P(DEA-stat-TMA)] block copolymers, poly(ethylene glycol) (PEG) crosslinked glycol chitosan (GC), Pluronic copolymers, poly(N,N-diethyl acrylamide) (pNNDEA), or the like. In various embodiments, polymers for nucleic acid delivery includes, but not limited to, PS, poly(lactic acid) (PLA), poly(lactic-co-glycolic acid) (PLGA), and polyplexes of cationic polymers, polyplexes of reporter gene DNA and polyethyleneimine (PEI), poly(l-lysine)/DNA (PLL/DNA), the like, or combination thereof. In various embodiments, therapeutic agents are capable of being released passively or actively from said encapsulations by one or more energy or motive producing modality, including but not limited to, a change in magnitude, frequency, intensity, power, potential, kinetic, gradient, and or reaction of a chemical, pH, thermal, electric, electrophoretic, magnetic, magneto dynamic, electromagnetic, catalytic, piezo-catalytic, or ultrasonic energy or motive producing modality.

### Administering a Therapeutic Agent

### Methods of Administration

Methods of the invention can include administering a therapeutic agent to gastrointestinal tissue of a subject using the systems and devices described above. The methods can include delivering ultrasound energy to a liquid at a frequency that produces bubbles within the liquid and causes transient cavitation of the bubbles. Gentle implosion of the bubbles produces shock waves that permeabilize cells and propel the agent from the liquid into the tissue. The use of ultrasound to cause transient cavitation to deliver agents to tissue is described in, for example, Schoellhammer, C. M., Schroeder, A., Maa, R., Lauwers, G. Y., Swiston, A., Zervas, M., et al. (2015). Ultrasound-mediated gastrointestinal drug delivery. Science Translational Medicine, 7(310), 310ra168-310ra168, doi: 10.1126/scitranslmed.aaa5937; Schoellhammer, C. M & Traverso, G., Low-frequency ultrasound for drug delivery in the gastrointestinal tract. Expert Opinion on Drug Delivery, 2016, doi: 10.1517/17425247.2016.1171841; Schoellhammer C. M., et al., Ultrasound-mediated delivery of RNA to colonic mucosa of live mice, Gastroenterology, 2017, doi: 10.1053/j.gastro.2017.01.002; and U.S. Publication Nos. 2014/0228715 and 2018/0055991, the contents of each of which are incorporated herein by reference.

In methods of the invention, the ultrasound signal may have a defined frequency. The ultrasound signal may have a frequency of from about 10 kHz to about 10 MHz, from about 10 kHz to about 1 MHz, from about 10 kHz to about 100 kHz, from about 20 kHz to about 80 kHz, from about 20 kHz to about 60 kHz, or from about 30 kHz to about 50 kHz. The ultrasound signal may have a frequency of less than 100 kHz, less than 80 kHz, less than 60 kHz, or less than 50 kHz. The ultrasound signal may have a frequency of about 20 kHz, about 25 kHz, about 30 kHz, about 35 kHz, about 40 kHz, about 45 kHz, about 50 kHz, about 55 kHz, or about 60 kHz.

In methods of the invention, the ultrasound signal may have a defined intensity. For example, and without limitation, the ultrasound signal may have an intensity of from about 0.1 W/cm² to about 10 W/cm², from about 0.24 W/cm² to about 1.4 W/cm², from about 1.4 W/cm² to about 10 W/cm², from about 10 W/cm² to about 100 W/cm², from about 100 W/cm² to about 500 W/cm², or from about 500 W/cm² to about 1000 W/cm².

In some embodiments, the ultrasound energy may be delivered as a pulse, i.e., it may be delivered over a brief, finite period to minimize damage to the agent being delivered by the ultrasound energy. For example, and without limitation, the pulse may be less than 20 minutes, less than 10 minutes, less than 5 minutes, or less than 10 minutes. For example, and without limitation, the pulse may be from about 10 seconds to about 3 minutes. The pulse may be about 10 minutes, about 5 minutes, about 3 minutes, about 3 minutes, about 1 minute, about 30 seconds, about 20 seconds, or about 10 seconds.

The parameters of the ultrasound pulse, such as the frequency and/or duration, may be selected so that damage to the agent is limited to a certain fraction or percentage of the agent. For example, and without limitation, the ultrasound energy may result in breakdown of less than about 95% of the agent, less than about 90% of the agent, less than about 80% of the agent, less than about 70% of the agent, less than about 60% of the agent, less than about 50% of the agent, less than about 40% of the agent, less than about 25% of the agent, or less than about 10% of the agent.

The parameters of the ultrasound pulse, such as the frequency and/or duration, may be selected so that at least a minimum amount of the agent is transferred to the tissue. For example, and without limitation, the ultrasound energy may result in transfer of at least 1% of the agent, at least 2% of the agent, at least 5% of the agent, at least 10% of the agent, at least 20% of the agent, at least 30% of the agent, or at least 40% of the agent.

The methods may be used to deliver a therapeutic agent to a specific tissue in the GI tract. For example, the tissue may be buccal tissue, gingival tissue, labial tissue, esophageal tissue, gastric tissue, intestinal tissue, colorectal tissue, or anal tissue. The therapeutic agent may be targeted to a particular tissue in the GI tract. For example, the therapeutic agent may be targeted to the stomach, small intestine, large intestine (colon), rectum, or at a duct that enters the GI tract, such as a pancreatic duct or a common bile duct.

The methods may include administering an ingestible capsule to the subject. The ingestible capsule may be administered orally or rectally. The ingestible capsule may be administered via a duct that enters the GI tract.

The methods may include positioning the ingestible capsule within the subjects GI tract. For example, the ingestible capsule may be positioned in proximity to an affected region of the GI tract, such as an ulcer or inflamed region. The ingestible capsule may be positioned by applying a magnetic field to a portion of the subject's GI tract from a device outside the subject's body. The magnetic field may be applied using the transmitter. Alternatively, or additionally, the magnetic field may be applied from a magnetic device that is separate from the transmitter.

### Therapeutic Agents

The therapeutic agent may be any agent that provides a therapeutic benefit. For example and without limitation, suitable agents include alpha-hydroxy formulations, ace inhibiting agents, analgesics, anesthetic agents, anthelmintics, anti-arrhythmic agents, antithrombotic agents, anti-allergic agents, anti-angiogenic agents, antibacterial agents, antibiotic agents, anticoagulant agents, anticancer agents, antidiabetic agents, anti-emetics, antifungal agents, antigens, antihypertension agents, antihypotensive agents, antiinflammatory agents, antimicotic agents, antimigraine agents, anti-obesity agents, antiparkinson agents, antirheumatic agents, antithrombins, antiviral agents, antidepressants, antiepileptics, antihistamines, antimuscarinic agents, antimycobacterial agents, antineoplastic agents, antithyroid agents, anxiolytics, asthma therapies, astringents, beta blocking agents, blood products and substitutes, bronchospamolytic agents, calcium antagonists, cardiovascular agents, cardiac glycosidic agents, carotenoids, cephalosporins, chronic bronchitis therapies, chronic obstructive pulmonary disease therapies, contraceptive agents, corticosteroids, cytostatic agents, cystic-fibrosis therapies, cardiac inotropic agents, contrast media, cough suppressants, diagnostic agents, diuretic agents, dopaminergics, elastase inhibitors, emphysema therapies, enkephalins, fibrinolytic agents, growth hormones, hemostatics, immunological agents, immunosupressants, immunotherapeutic agents, insulins, interferons, lactation inhibiting agents, lipid-lowering agents, lymphokines, muscle relaxants, neurologic agents, NSAIDS, nutraceuticals, oncology therapies, organ-transplant rejection therapies, parasympathomimetics, parathyroid calcitonin and biphosphonates, prostacyclins, prostaglandins, psycho-pharmaceutical agents, protease inhibitors, magnetic resonance diagnostic imaging agents, radio-pharmaceuticals, reproductive control hormones, respiratory distress syndrome therapies, sedative agents, sex hormones, somatostatins, steroid hormonal agents, stimulants and anoretics, sympathomimetics, thyroid agents, vasodilating agents, vitamins, and xanthines. A more complex list of chemicals and drugs that can be used as agents in embodiments of the invention is provided in the Merck Index: An Encyclopedia of Chemicals, Drugs, and Biologicals Fifteenth Edition, Maryadele J O'Neil, ed., RSC Publishing, 2015, ISBN-13: 978-1849736701, ISBN-10 1849736707, the contents of which are incorporated herein by reference.

Therapeutic agents may be of any chemical form. For example, agents may be biological therapeutics, such as nucleic acids, proteins, peptides, polypeptides, antibodies, or other macromolecules. Nucleic acids include RNA, DNA, RNA/DNA hybrids, and nucleic acid derivatives that include non-naturally-occurring nucleotides, modified nucleotides, non-naturally-occurring chemical linkages, and the like. Examples of nucleic acid derivatives and modified nucleotides are described in, for example, International Publication WO 2018/118587, the contents of which are incorporated herein by reference. Nucleic acids may be polypeptide-encoding nucleic acids, such as mRNAs and cDNAs. Nucleic acids may interfere with gene expression. Examples of interfering RNAs (RNAi) include siRNAs and miRNAs. RNAi is known in the art and described in, for example, Kim and Rossi, Biotechniques. 2008 April; 44(5): 613-616, doi: 10.2144/000112792; and Wilson and Doudna, Molecular Mechanisms of RNA Interference, Annual Review of Biophysics 2013 42:1, 217-239, the contents of each of which are incorporated herein by reference. Agents may be organic molecules of non-biological origin. Such drugs are often called small-molecule drugs because they typically have a molecular weight of less than 2000 Daltons, although they may be larger. Agents may be combinations or complexes of one or more biological macromolecules and/or one or more small molecules. For example and without limitation, agents may be nucleic acid complexes, protein complexes, protein-nucleic acid complexes, and the like. Thus, the agent may exist in a multimeric or polymeric form, including homocomplexes and heterocomplexes.

An advantage of ultrasound-based delivery of therapeutic agents is the capacity to deliver large molecules, e.g., molecules having a molecular weight greater than 1000 Da. Thus, the therapeutic agent may have a minimum size. For example and without limitation, the antigen may have a molecular weight of >100 Da, >200 Da, >500 Da, >1000 Da, >2000 Da, >5000 Da, >10,000 Da, >20,000 Da, >50,000 Da, or >100,000 Da.

The therapeutic agent may be provided in a liquid that promotes delivery of the therapeutic agent using the devices or systems provided herein. For example, the liquid may facilitate ultrasound-induced cavitation, iontophoresis, sonoporation, magnetosonoporation, or electroporation. The liquid may be aqueous. The liquid may contain ions. The liquid may be an aqueous solution that contains one or more salts. The liquid may contain a buffer.

The therapeutic agent may be formulated. Formulations commonly used for delivery of biologic and small-molecule agents include drug crystals, gold particles, iron oxide particles, lipid-like particles, liposomes, micelles, microparticles, nanoparticles, polymeric particles, vesicles, viral capsids, viral particles, and complexes with other macromolecules that are not essential for the biological or biochemical function of the agent.

Alternatively, the therapeutic agent may be unformulated, i.e., it may be provided in a biologically active format that does not contain other molecules that interact with the agent solely to facilitate delivery of the agent. Thus, the agent may be provided in a non-encapsulated form or in a form that is not complexed with other molecules unrelated to the function of the agent.

The agent may be a component of a gene editing system, such as a meganuclease, zinc finger nuclease (ZFN), a transcription activator-like effector-based nuclease (TALEN), or the clustered, regularly-interspersed palindromic repeat (CRISPR) system.

Meganucleases are endodeoxyribonucleases that recognize double-stranded DNA sequences of 12-40 base pairs. They can be engineered to bind to different recognition sequences to create customized nucleases that target sequences. Meganucleases exist in archaebacterial, bacteria, phages, fungi, algae, and plants, and meganucleases from any source may be used. Engineering meganucleases to recognize specific sequences is known in the art and described in, for example, Stoddard, Barry L. (2006) “Homing endonuclease structure and function” Quarterly Reviews of Biophysics 38 (1): 49-95 doi: 10.1017/S0033583505004063, PMID 16336743; Grizot, S.; Epinat, J. C.; Thomas, S.; Duclert, A.; Rolland, S.; Paques, F.; Duchateau, P. (2009) “Generation of redesigned homing endonucleases comprising DNA-binding domains derived from two different scaffolds” Nucleic Acids Research 38 (6): 2006-18, doi: 10.1093/nar/gkp1171. PMC 2847234, PMID 20026587; Epinat, Jean-Charles; Arnould, Sylvain; Chames, Patrick; Rochaix, Pascal; Desfontaines, Dominique; Puzin, Clémence; Patin, Amélie; Zanghellini, Alexandre; Pâques, Frédéric (2003-06-01) “A novel engineered meganuclease induces homologous recombination in yeast and mammalian cells” Nucleic Acids Research 31 (11): 2952-2962; and Seligman, L. M.; Chisholm, K M; Chevalier, B S; Chadsey, M S; Edwards, S T; Savage, J H; Veillet, A L (2002) “Mutations altering the cleavage specificity of a homing endonuclease” Nucleic Acids Research 30 (17): 3870-9, doi:10.1093/nar/gkf495. PMC 137417, PMID 12202772, the contents of each of which are incorporated herein by reference.

ZFNs are artificial restriction enzymes that have a zinc finger DNA-binding domain fused to a DNA-cleavage domain. ZFNs can also be engineered to target specific DNA sequences. The design and use of ZFNs is known in the art and described in, for example, Carroll, D (2011) “Genome engineering with zinc-finger nucleases” Genetics Society of America 188 (4): 773-782, doi: 10.1534/genetics. 111.131433. PMC 3176093, PMID 21828278; Cathomen T, Joung J K (July 2008) “Zinc-finger nucleases: the next generation emerges” Mol. Ther. 16 (7): 1200-7, doi:10.1038/mt.2008.114, PMID 18545224; Miller, J. C.; Holmes, M. C.; Wang, J.; Guschin, D. Y.; Lee, Y. L.; Rupniewski, I.; Beausejour, C. M.; Waite, A. J.; Wang, N. S.; Kim, K. A.; Gregory, P. D.; Pabo, C. O.; Rebar, E. J. (2007) “An improved zinc-finger nuclease architecture for highly specific genome editing” Nature Biotechnology, 25 (7): 778-785, doi: 10.1038/nbt1319, PMID 17603475, the contents of each of which are incorporated herein by reference.

TALENs are artificial restriction enzymes that have a TAL effector DNA-binding domain fused to a DNA cleavage domain. TALENs can also be engineered to target specific DNA sequences. The design and use of TALENs is known in the art and described in, for example, Boch J (February 2011) “TALEs of genome targeting” Nature Biotechnology 29 (2): 135-6, doi:10.1038/nbt.1767. PMID 21301438; Juillerat A, Pessereau C, Dubois G, Guyot V, Maréchal A, Valton J, Daboussi F, Poirot L, Duclert A, Duchateau P (January 2015) “Optimized tuning of TALEN specificity using non-conventional RVDs” Scientific Reports, 5: 8150, doi: 10.1038/srep08150. PMC 4311247, PMID 25632877; and Mahfouz M M, Li L, Shamimuzzaman M, Wibowo A, Fang X, Zhu J K (February 2011) “De novo-engineered transcription activator-like effector (TALE) hybrid nuclease with novel DNA binding specificity creates double-strand breaks” Proceedings of the National Academy of Sciences of the United States of America, 108 (6): 2623-8, Bibcode:2011PNAS, 108.2623M, doi: 10.1073/pnas. 1019533108, PMC 3038751, PMID 21262818, the contents of each of which are incorporated herein by reference.

The CRISPR system is a prokaryotic immune system that provides acquired immunity against foreign genetic elements, such as plasmids and phages. CRISPR systems include one or more CRISPR-associated (Cas) proteins that cleave DNA at clustered, regularly-interspersed palindromic repeat (CRISPR) sequences. Cas proteins include helicase and exonuclease activities, and these activities may be on the same polypeptide or on separate polypeptides. Cas proteins are directed to CRISPR sequences by RNA molecules. A CRISPR RNA (crRNA) binds to a complementary sequence in the target DNA to be cleaved. A transactivating crRNA (tracrRNA) binds to both the Cas protein and the crRNA to draw the Cas protein to the target DNA sequence. Not all CRISPR systems require tracrRNA. In nature crRNA and tracrRNA occur on separate RNA molecules, but they also function when contained a single RNA molecule, called a single guide RNA or guide RNA (gRNA). The one or more RNAs and one or more polypeptides assemble inside the cell to form a ribonucleoprotein (RNP). CRISPR systems are described, for example, in van der Oost, et al., CRISPR-based adaptive and heritable immunity in prokaryotes, Trends in Biochemical Sciences, 34(8):401-407 (2014); Garrett, et al., Archaeal CRISPR-based immune systems: exchangeable functional modules, Trends in Microbiol. 19(11):549-556 (2011); Makarova, et al., Evolution and classification of the CRISPR-Cas systems, Nat. Rev. Microbiol. 9:467-477 (2011); and Sorek, et al., CRISPR-Mediated Adaptive Immune Systems in Bacteria and Archaea, Ann. Rev. Biochem. 82:237-266 (2013), the contents of each of which are incorporated herein by reference.

CRISPR-Cas systems have been placed in two classes. Class 1 systems use multiple Cas proteins to degrade nucleic acids, while class 2 systems use a single large Cas protein. Class 1 Cas proteins include Cas10, Cas10d, Cas3, Cas5, Cas8a, Cmr5, Cse1, Cse2, Csf1, Csm2, Csx11, Csy1, Csy2, and Csy3. Class 2 Cas proteins include C2c1, C2c2, C2c3, Cas4, Cas9, Cpf1, and Csn2.

CRISPR-Cas systems are powerful tools because they allow gene editing of specific nucleic acid sequences using a common protein enzyme. By designing a guide RNA complementary to a target sequence, a Cas protein can be directed to cleave that target sequence. In addition, although naturally-occurring Cas proteins have endonuclease activity, Cas proteins have been engineered to perform other functions. For example, endonuclease-deactivated mutants of Cas9 (dCas9) have been created, and such mutants can be directed to bind to target DNA sequences without cleaving them. dCas9 proteins can then be further engineered to bind transcriptional activators or inhibitors. As a result, guide sequences can be used to recruit such CRISPR complexes to specific genes to turn on or off transcription. Thus, these systems are called CRISPR activators (CRISPRa) or CRISPR inhibitors (CRISPRi). CRISPR systems can also be used to introduce sequence-specific epigenetic modifications of DNA, such acetylation or methylation. The use of modified CRISPR systems for purposes other than cleavage of target DNA are described, for example, in Dominguez, et al., Beyond editing: repurposing CRISPR-Cas9 for precision genome regulation and interrogation, Nat. Rev. Cell Biol. 17(1):5-15 (2016), which is incorporated herein by reference.

The agent may be any component of a CRISPR system, such as those described above. For example and without limitation, the CRISPR component may be one or more of a helicase, endonuclease, transcriptional activator, transcriptional inhibitor, DNA modifier, gRNA, crRNA, or tracrRNA. The CRISPR component contain a nucleic acid, such as RNA or DNA, a polypeptide, or a combination, such as a RNP. The CRISPR nucleic acid may encode a functional CRISPR component. For example, the nucleic acid may be a DNA or mRNA. The CRISPR nucleic acid may itself be a functional component, such as a gRNA, crRNA, or tracrRNA.

The agent may include an element that induces expression of the CRISPR component. For example, expression of the CRISPR component may be induced by an antibiotic, such as tetracycline, or other chemical. Inducible CRISPR systems have been described, for example, in Rose, et al., Rapidly inducible Cas9 and DSB-ddPCR to probe editing kinetics, Nat. Methods, 14, pages 891-896 (2017); and Cao, et al., An easy and efficient inducible CRISPR/Cas9 platform with improved specificity for multiple gene targeting, Nucleic Acids Res. 14(19): e149 (2016), the contents of which are incorporated herein by reference. The inducible element may be part of the CRISPR component, or it may be a separate component.

In certain embodiments of the invention, methods allow delivery of agents that promote wound healing. The agent may promote healing by any mechanism. For example and without limitation, the agent may facilitate one or more phases of the wound healing process; prevent infection, including bacterial or viral infection; or alleviate pain or sensitivity.

A variety of growth factors promote wound healing. For example and without limitation, growth factors that promote wound healing include CTGF/CCN2, EGF family members, FGF family members, G-CSF, GM-CSF, HGF, HGH, HIF, histatin, hyaluronan, IGF, IL-1, IL-4, IL-8, KGF, lactoferrin, lysophosphatidic acid, NGF, a PDGF, TGF-β, and VEGF. The EFG family includes 10 members: amphiregulin (AR), betacellulin (BTC), epigen, epiregulin (EPR), heparin-binding EGF-like growth factor (HB-EGF), neuregulin-1 (NRG1), neuregulin-2 (NRG2), neuregulin-3 (NRG3), neuregulin-4 (NRG4), or transforming growth factor-α (TGF-α). The FGF family includes 22 members: FGF1, FGF2 (also called basic FGF or bFGF), FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, or FGF23. PDGF exists in three forms: PDGF AA, PDGF AB, and PDGF BB. The TGF-family includes three forms: TGF-β1, TGF-β2, and TGF-β3.

A variety of agents that prevent infection have been used to treat wounds. For example, and without limitation, the agent may be an antimicrobial, antiviral, antibiotic, antifungal, or antiseptic. Exemplary agents include silver, iodine, chlorhexidine, hydrogen peroxide, lysozyme, peroxidase, defensins, cystatins, thrombospondin, and antibodies. Nitric oxide donors, such as glyceryl trinitrate and nitrite salts, are also useful to prevent infection and promote wound healing.

### Diseases, Disorders, and Conditions

The methods are useful to treat conditions of the GI tract of a subject. The condition may be any disease, disorder, or condition that affects the GI tract.

In some embodiments, the disorder is a disorder of the esophagus, including, but not limited to, esophagitis—(candidal), gastroesophageal reflux disease (gerd); laryngopharyngeal reflux (also known as extraesophageal reflux disease/eerd); rupture (Boerhaave syndrome, Mallory-Weiss syndrome); UES—(Zenker's diverticulum); LES—(Barrett's esophagus); esophageal motility disorder—(nutcracker esophagus, achalasia, diffuse esophageal spasm); esophageal stricture; and megaesophagus.

In some embodiments, the disorder is a disorder of the stomach, including but not limited to gastritis (e.g., atrophic, Menetrier's disease, gastroenteritis); peptic (i.e., gastric) ulcer (e.g., Cushing ulcer, Dieulafoy's lesion); dyspepsia; emesis; pyloric stenosis; achlorhydria; gastroparesis; gastroptosis; portal hypertensive gastropathy; gastric antral vascular ectasia; gastric dumping syndrome; and human mullular fibrillation syndrome (HMFS).

In some embodiments, the disorder is a disorder of the small intestine, including but not limited to, enteritis (duodenitis, jejunitis, ileitis); peptic (duodenal) ulcer (curling's ulcer); malabsorption: celiac; tropical sprue; blind loop syndrome; Whipple's; short bowel syndrome; steatorrhea; milroy disease In some embodiments, the disorder is a disorder of the small intestine, including but not limited to, both large intestine and small intestine enterocolitis (necrotizing); inflammatory bowel disease (IBD); Crohn's disease; vascular; abdominal angina; mesenteric ischemia; angiodysplasia; bowel obstruction: ileus; intussusception; volvulus; fecal impaction; constipation; and diarrhea.

In some embodiments, the disorder is a disorder of the small intestine, including but not limited to, accessory digestive glands disease; liver hepatitis (viral hepatitis, autoimmune hepatitis, alcoholic hepatitis); cirrhosis (PBC); fatty liver (Nash); vascular (hepatic veno-occlusive disease, portal hypertension, nutmeg liver); alcoholic liver disease; liver failure (hepatic encephalopathy, acute liver failure); liver abscess (pyogenic, amoebic); hepatorenal syndrome; peliosis hepatis; hemochromatosis; and Wilson's disease.

In some embodiments, the disorder is a disorder of the pancreas, including, but not limited to, pancreas pancreatitis (acute, chronic, hereditary); pancreatic pseudocyst; and exocrine pancreatic insufficiency.

In some embodiments, the disorder is a disorder of the large intestine, including but not limited to, appendicitis; colitis (pseudomembranous, ulcerative, ischemic, microscopic, collagenous, lymphocytic); functional colonic disease (IBS, intestinal pseudoobstruction/ogilvie syndrome); megacolon/toxic megacolon; diverticulitis; and diverticulosis.

In some embodiments, the disorder is a disorder of the large intestine, including but not limited to, gall bladder and bile ducts, cholecystitis; gallstones/cholecystolithiasis; cholesterolosis; Rokitansky-Aschoff sinuses; postcholecystectomy syndrome cholangitis (PSC, ascending); cholestasis/Mirizzi's syndrome; biliary fistula; haemobilia; and gallstones/cholelithiasis. In some embodiments, the disorder is a disorder of the common bile duct (including choledocholithiasis, biliary dyskinesia).

Other disorders which can be treated with the methods and devices included herein include acute and chronic immune and autoimmune pathologies, such as systemic lupus erythematosus (SLE), rheumatoid arthritis, thyroidosis, graft versus host disease, scleroderma, diabetes mellitus, Graves' disease, Beschet's disease; inflammatory diseases, such as chronic inflammatory pathologies and vascular inflammatory pathologies, including chronic inflammatory pathologies such as sarcoidosis, chronic inflammatory bowel disease, ulcerative colitis, and Crohn's pathology and vascular inflammatory pathologies, such as, but not limited to, disseminated intravascular coagulation, atherosclerosis, giant cell arteritis and Kawasaki's pathology; malignant pathologies involving tumors or other malignancies, such as, but not limited to leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or Mycosis fungoides)); carcinomas (such as colon carcinoma) and metastases thereof; cancer-related angiogenesis; infantile haemangiomas; and infections, including, but not limited to, sepsis syndrome, cachexia, circulatory collapse and shock resulting from acute or chronic bacterial infection, acute and chronic parasitic and/or infectious diseases, bacterial, viral or fungal, such as a HIV, AIDS (including symptoms of cachexia, autoimmune disorders, AIDS dementia complex and infections).

Other disorders which can be treated with the methods and devices included herein include acute and chronic immune and autoimmune pathologies, inflammatory diseases, infections and malignant pathologies involving, e.g., tumors or other malignancies.

The subject suffering from the GI condition may be any type of subject, such as an animal, for example, a mammal, for example, a human.

It should be appreciated that all combinations of the concepts discussed in greater detail below (provided such concepts are not mutually inconsistent) are contemplated as being part of the inventive subject matter disclosed herein. It also should be appreciated that terminology explicitly employed herein that also may appear in any disclosure incorporated by reference should be accorded a meaning most consistent with the concepts disclosed herein.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. The present disclosure should in no way be limited to the exemplary implementation and techniques illustrated in the drawings and described below.

Where a range of values is provided, it is understood that each intervening value, to the tenth of the unit of the lower limit unless the context clearly dictates otherwise, between the upper and lower limit of that range and any other stated or intervening value in that stated range is encompassed by the invention. The upper and lower limits of these smaller ranges may independently be included in the smaller ranges, and are also encompassed by the invention, subject to any specifically excluded limit in a stated range. Where a stated range includes one or both endpoint limits, ranges excluding either or both of those included endpoints are also included in the scope of the invention.

As used herein, the term “includes” means includes but is not limited to, the term “including” means including but not limited to. The term “based on” means based at least in part on.

The term transducer, as used herein, may refer to a device that converts energy from one form to another.

The term Helmholtz coil, as used herein, may refer to a device for producing a region of nearly uniform magnetic field, named after the German physicist Hermann von Helmholtz. It consists of two electromagnets on the same axis.

The term load, as used herein, may refer to a device connected to a signal source, whether or not it consumes power.

The term electroporation, as used herein, may refer to a method or technique in which an electrical field is applied to cells to increase the permeability of the cell membrane, allowing chemicals, drugs, or nucleic acid to be introduced into the cell.

The term sonoporation, as used herein, may refer to the use of sound (typically ultrasonic frequencies) for modifying the permeability of the cell plasma membrane. Sonoporation employs the acoustic cavitation of microbubbles to enhance delivery of small and large molecules.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. An ingestible capsule comprising:
an inductive receiver coil configured to receive an electromagnetic signal from a transmitter external to the capsule;
an ultrasound transducer electrically coupled to the inductive receiver; and
a reservoir configured to releasably retain a liquid comprising a therapeutic agent, wherein the capsule does not comprise a power source.

2. The ingestible capsule of claim 1, wherein the ultrasound transducer is positioned to transduce ultrasound waves toward the reservoir.

3. The ingestible capsule of claim 1, wherein the ultrasound transducer is positioned to transduce ultrasound waves away from the reservoir.

4. The ingestible capsule of claim 1, wherein the capsule does not comprise a component that alters a frequency of an electromagnetic signal received by the inductive receiver coil.

5. The ingestible capsule of claim 1, further comprising:
a modulator electrically coupled to the inductive receiver coil and the ultrasound transducer, the modulator configured to modulate a frequency of an electromagnetic signal received by the inductive receiver coil.

6. The ingestible capsule of claim 5, wherein the modulator is a multiplier configured to increase the frequency of the electromagnetic signal received by the inductive receiver coil.

7. The ingestible capsule of claim 5, wherein the modulator is an attenuator configured to decrease the frequency of the electromagnetic signal received by the inductive receiver coil.

8. The ingestible capsule of claim 1, wherein the ultrasound transducer is configured to produce an ultrasound signal at a frequency of from about 20 kHz to about 60 kHz.

9. The ingestible capsule of claim 1, wherein a longest dimension of the capsule is not greater than about 2.5 cm.

10. The ingestible capsule of claim 1, further comprising:
a rectifier electrically coupled to the inductive receiver; and
an electrode electrically coupled to the rectifier and in contact with the reservoir.

11. A system comprising:
a transmitter comprising:
a power source; and
a transmitter coil electrically coupled to the power source; and
an ingestible capsule that is physically separate from the transmitter, the ingestible capsule comprising:
an inductive receiver coil configured to receive an electromagnetic signal from the transmitter when the transmitter is not in contact with the ingestible capsule;
an ultrasound transducer electrically coupled to the inductive receiver; and
a reservoir configured to releasably retain a liquid comprising a therapeutic agent.

12. The transmitter of claim 11, wherein the power source, when activated, produces a DC voltage of from about 3.2 VDC to about 32 VDC.

13. The system of claim 12, wherein the transmitter further comprises:
a DC-DC converter downstream of the power source and upstream of the transmitter coil,
wherein when the when the power source is activated, the DC-DC converter increase the voltage produced by the power source to from about 16 VDC to about 160 VDC.

14. The system of claim 13, wherein the DC-DC converter is connected directly to the transmitter coil without intervening components.

15. The system of claim 13, wherein the transmitter further comprises:
a voltage-controlled oscillator downstream of the DC-DC converter;
a FET driver downstream of the DC-DC converter;
a FET transistor network downstream of the FET driver; and
a capacitor downstream of the FET driver; and
a resistor downstream of the FET transistor.

16. The system of claim 11, wherein the transmitter is configured to be held in a hand of a person.

17. The system of claim 11, wherein the transmitter comprises a wearable garment.

18. The system of claim 11, wherein the power source is rechargeable.

19. The system of claim 11, wherein the transmitter comprises at least one of:
a user interface configured to receive input from a user, and
a display.

20. The system of claim 11, wherein the transmitter comprises a microprocessor configured to communicate with a device external to the system.

21. A method of administering a therapeutic agent to a gastrointestinal tissue of a subject, the method comprising:
orally administering to a subject an ingestible capsule that does not comprise a power source, the capsule comprising:
an inductive receiver coil;
an ultrasound transducer electrically coupled to the inductive receiver; and
a reservoir comprising a liquid comprising a therapeutic agent, and
transmitting via a transmitter external to the subject an electromagnetic signal to the ingestible capsule to allow the ultrasound transducer to generate an ultrasound signal and thereby deliver the therapeutic agent from the reservoir into gastrointestinal tissue of the subject.

22. The method of claim 21, wherein the transmitter comprises:
a power source; and
a transmitter coil electrically coupled to the power source; and
wherein the electromagnetic signal is transmitted from the transmitter coil to the inductive receiver coil.

23. The method of claim 21, wherein the ingestible capsule further comprises:
a rectifier electrically coupled to the inductive receiver; and
an electrode electrically coupled to the rectifier and in contact with the reservoir.

24. The method of claim 23, wherein the transmission of the electromagnetic signal generates an electrical signal in the liquid that promotes movement of the therapeutic agent from the reservoir and into the gastrointestinal tissue.

25. The method of claim 24, wherein the electrical signal is a DC signal or a DC pulse train.

26. The method of claim 24, wherein the electrical signal promotes movement of the therapeutic agent by iontophoresis or electroporation.

27. The method of claim 21, wherein the transmitter generates a magnetic field that positions the ingestible capsule adjacent to the gastrointestinal tissue of the subject.

28. The method of claim 21, wherein a frequency of the electromagnetic signal is about equal to a frequency of the ultrasound signal.

29. The method of claim 21, wherein a frequency of the electromagnetic signal is not equal to a frequency of the ultrasound signal.

30. The method of claim 21, wherein the ultrasound signal has a frequency of from about 20 kHz to about 60 KHz.
